(19) United States
(12) Patent Application Publication
(10) Pub. No.: US 2024/0398973 A1
(43) Pub. Date: Dec. 5, 2024

(54) ENGINEERED ANTIBODY COMPOUNDS AND CONJUGATES THEREOF

(71) Applicant: Eli Lilly and Company, Indianapolis, IN (US)

(72) Inventors: Michael James Bacica, San Diego, CA (US); Yiqing Feng, Carmel, IN (US); Donmienne Doen Mun Leung, San Diego, CA (US); Matthew D. Linnik, Solana Beach, CA (US); Adam Robert Mezo, San Diego, CA (US); James Thomas Parker, San Diego, CA (US); Purva Vivek Trivedi, Whitestown, IN (US); Francisco Alcides Valenzuela, Indianapolis, IN (US); Jianghuai Xu, San Diego, CA (US)

(21) Appl. No.: 18/808,584

(22) Filed: Aug. 19, 2024

Related U.S. Application Data
(63) Continuation of application No. 16/619,846, filed on Dec. 5, 2019.
(60) Provisional application No. 62/520,855, filed on Jun. 16, 2017.

(51) Int. Cl. A61K 47/68; A61K 35/00; C07K 16/32

(52) U.S. Cl. CPC A61K 47/6889; A61K 35/00; A61K 47/6811; A61K 47/6849; A61K 47/6855; A61K 47/6857; A61K 47/6861; A61K 47/6863; A61K 47/6865; A61K 47/6869; C07K 16/32

(57) ABSTRACT

Engineered antibody compounds and conjugates thereof, are provided, said antibody compounds and conjugates thereof are useful as agents for cancer immunotherapy. Engineered antibody compounds may include one or more amino acid residue substitutions within the constant regions of the antibody engineered into a parental antibody. Amino acid residue substitutions may include substitution of cysteine or alanine.

### CROSS-RELATED APPLICATIONS

This application is a continuation of U.S. patent application Ser. No. 16/619,846 filed Dec. 5, 2019, which is the US National Stage of International Application No. PCT/US2018/037495 filed Jun. 14, 2018 which claims the benefit of U.S. Provisional Application No. 62/520,855 filed Jun. 16, 2017. The foregoing applications are incorporated herein by reference.

### SEQUENCE LISTING

The instant application contains a Sequence Listing which has been submitted electronically in XML file format and is hereby incorporated by reference in its entirety. Said XML copy, created on Aug. 16, 2024, is named X20828A_44P58WOUS2_SL.xml and is 119,698 bytes in size.

The present invention relates to novel antibody compounds and methods of use thereof.

Antibodies, and truncated fragments thereof may be conjugated with a variety of payloads including therapeutic, cytotoxic, and diagnostic peptides or other small molecules, for in vivo and in vitro applications. Antibody conjugates may be synthesized using free cysteine sulfhydryl groups, generated on the surface of immunoglobulin heavy chain or light chain residues, as reactive nucleophiles to form stable chemical linkages with the payload via a variety of linkers. However, conventional thiol-conjugation following the reduction of inter-chain disulfide bonds leads to a heterogeneous antibody-drug conjugate mixture depending on the reaction conditions. Even carefully controlled reactions will result in a distribution of the conjugate to antibody ratio (CR). Conjugate mixtures with higher CRs will display different chemical and biophysical characteristics compared to conjugate mixtures with a lower CR. Addition of payload to antibody can also alter the pharmacological properties of the antibody, including potentially impacting target binding and Fc receptor interactions. It is therefore desirable to obtain conjugates with a more uniform and targeted distribution of the conjugate to antibody ratio.

To enable a more homogenous and targeted distribution of payload-conjugated antibodies, cysteine residues have been engineered into parental mAbs to facilitate site-directed conjugation of drug payloads via thiol-conjugation. (e.g. U.S. Pat. No. 7,521,541) However, mutation of a parental surface amino acid residue to a cysteine may impact mAb biophysical properties and expression. For example, the engineered cysteine residue could disrupt native disulfides which are critical for proper protein folding. Further, the resulting unpaired cysteine could also form intermolecular disulfides, resulting in high order aggregates. Thus, there remains a need for further IgG mAbs comprising alternative engineered-cysteine residues. There also remains a need for such antibodies in a compound that engages the cells of the immune system.

Cancer immunotherapy harnesses the body's immune system to attack cancer cells and is a dynamic area in oncology drug discovery and development. The therapeutic approaches represent a paradigm shift to engage the host's immune system to recognize and destroy tumor cells, in contrast to therapies based on the use of tumoricidal agents. Two successful cancer immunotherapy strategies are inhibiting suppression of the immune system to enable activation of adaptive and/or innate immune system, especially tumor-directed cytotoxic T-cells (i.e., immune checkpoint blockade), and antibody modifications designed to engage and/or enhance antibody-dependent cell-mediated cytotoxicity (ADCC).

Successful clinical outcomes have recently been achieved with immune checkpoint modulators designed to modify interactions between T-cell surface receptors, such as PD-1 and CTLA-4, and cognate ligand in a manner that results in activation of the T-cells and resulting in T-cell mediated tumor cell destruction. Cancer immunotherapies targeting PD-1 (e.g., nivolumab (Opdivo®) and pembrolizumab (Keytruda®)) and CTLA-4 (e.g., ipilimumab (Yervoy®) have been FDA approved for the treatment of cancers such as squamous non-small cell lung cancer and metastatic melanoma.

ADCC involves interactions of antibody Fc domains with receptors (e.g., Fc gamma receptor IIIa) located on the surface of immune system cells (e.g., natural killer or “NK” cells) resulting in the release of cytolytic proteins from the immune cell with subsequent destruction of the targeted tumor cell. Approved antibody therapies displaying ADCC include Rituxin® (rituximab), Arzerra® (ofatumumab), Herceptin® (trastuzumab) and Campath® (alemtuzumab). Efforts to engineer antibodies with improved ADCC activity via enhanced Fc receptor binding have been effective in patients where antibodies with similar target specificity and less ADCC activation are ineffective or no longer adequately effective in the disease (e.g., Gazyva® (obinutuzumab)).

Notwithstanding progress in current cancer immunotherapies, there remains a need for alternative approaches to engage the immune system in treating cancer. For example, the percentage of patients that respond to T-cell directed immunotherapies varies and there is a lack of reliable prognostic assays that identify which patients will respond. In addition, therapy-induced autoimmune disease is a serious side effect associated with immune checkpoint inhibitor therapy. The emergence of autoimmune disease with immune checkpoint inhibitors is likely related to their mechanism of action as they are designed to remove suppression of the T-cell repertoire so that tumor-specific T-cells can emerge, proliferate and be activated. Thus, they are relatively non-specific, and one consequence of this lack of specificity is that it allows self-reactive T-cells to break tolerance and induce autoimmune disease which is not necessarily reversible on cessation of therapy. Enhanced ADCC approaches are designed to engage the NK cells for tumor cell killing. However, NK cells only constitute about 5% of the total leukocyte population in blood.

Targeting polymorphonuclear cells (PMNs) of the innate immune system to engage in tumor cell killing represents an alternative approach to cancer immunotherapy. PMNs comprise more than 50% of the total leukocyte population, and are a major line of defense against pathogens, including commensal and foreign bacteria. During the innate immune response, pathogen-associated molecular patterns (PAMPs) presented by the pathogen are recognized by pattern recognition receptors (PRRs) located on the surface of immune cells such as neutrophils. One such PRR is formyl peptide receptor 1 (FPR1), a membrane bound G-protein coupled receptor expressed on the neutrophil cell surface. FPR1 detects proteins and peptides with N-formyl-methionines including those produced and released by bacteria following infection. Engagement of FPR1 on the surface of neutrophils with N-formyl-Methionine-containing peptides, particularly those presenting N-formyl-methionine-leucine-phenylalanine (“fMLF” herein) residues, triggers motility/chemotaxis of neutrophils toward the site of infection. Activation of FPR1 by formyl peptides also elicits pathogen killing mechanisms such as degranulation to release cytotoxic molecules, production of reactive oxygen species and phagocytosis in order to destroy the pathogen. There are extensive descriptions of natural and non-natural FPR-1 agonists in the literature that are relevant to the current invention (He HQ and Ye RD, Molecules. 2017 Mar. 13; 22(3). pii: E455. doi: 10.3390/molecules22030455; Hwang TL et al., Org Biomol Chem. 2013 Jun. 14; 11(22):3742-55. doi: 10.1039/c3ob40215k; Cavicchioni G et al., Bioorg Chem. 2006 October; 34(5):298-318; Higgins JD et al., J Med Chem. 1996 Mar. 1; 39(5):1013-5; Vergelli C et al., Drug Dev Res. 2017 February; 78(1):49-62. doi: 10.1002/ddr.21370; Kirpotina LN et al., Mol Pharmacol. 2010 February; 77(2):159-70. doi: 10.1124/mol. 109.060673; Cilibrizzi A et al., J Med Chem. 2009 Aug. 27; 52(16):5044-57. doi: 10.1021/jm900592h.)

Prior efforts to utilize fMLF bioconjugates (antibody conjugated to a peptide) to attract macrophages to kill tumor cells encountered several limitations. Obrist and Sandberg conjugated fMLF to a polyclonal rabbit anti-tumor antibody using carbodiimide chemisty to link the peptide to free lysines. This non-specific conjugation of fMLF to polyclonal antibody led to a significant reduction in affinity, a 100-fold reduction in potency of fMLF for promoting macrophage chemotaxis, and a significantly dimished ability of the antibody to induce complement-dependent 51Cr release from pre-labeled hepatoma cells using normal rabbit serum as a complement source. (Obrist and Sandberg, Clin. Immun. Immunopathology, 25; 91-102 (1982)). These data are consistent with the possibility that non-specific addition of fMLF to antibody via lysine chemistry can reduce antigen binding affinity, FPR-1 receptor engagement, and Fc receptor engagement.

Obrist et al. showed that coupling fMLF to mouse monoclonal antibodies with carbodiimide chemistry allowed them to retain affinity for the human ovarian carcinoma cells, although the conjugation did reduce chemotactic response to human peripheral blood mononuclear cells. The impact of conjugation on complement fixation was not reported. (Obrist et al., Int. J. Immunopharmac., 5(4); 307-314 (1983)). Similar findings (preserved binding and impaired chemotaxis) were also reported when fMLF was conjugated directly to the melanoma mAb 9.2.27 via carbodiimide chemistry (Obrist et al., Caner Immunol. Immunother., 32; 406-08 (1991)). The antibody conjugate compounds of the present invention are capable of attracting and activating human neutrophils in addition to mononuclear cells and macrophages, whereas prior literature observations were almost exclusive directed to mononuclear cells and macrophages. This may have important therapeutic relevance, as neutrophils represent a greater percentage of the total white blood cell population in circulation in humans, are produced at a higher rate than all other leucocyte populations, can readily migrate into tissues, and are highly effective at eliminating target bacteria when activated.

The most common methods of antibody-drug conjugation are alkylation of reduced interchain disulfides, acylation of lysine residues, and alkylation of genetically engineered cysteine residues. The current invention contemplates that all common methods for generating antibody conjugates would be effective for producing an antibody conjugate capable of agonizing FPR-1 on neutrophils and cells of the innate immune system.

Tumor-targeting therapeutic antibodies capable of engaging PMN neutrophil cells of the innate immune system to participate in tumor cell destruction may also provide advantages over current cancer immunotherapies. For example, such a therapeutic antibody could enhance the T-cell response to the tumor, and may not require the presence of tumor-specific T-cells to drive tumor cell killing. Engagement of anti-tumor activity by PMN neutrophils would depend on the presence of FPRs (e.g., FPR1) which all patients would natively express on neutrophils. Further, an agent that is capable of engaging PMN neutrophils in tumor cell killing would benefit from a robust, continuous supply of tumor killing cells as it has been estimated that 1×10¹¹ neutrophils are produced per day. A tumor targeted antibody capable of engaging neutrophils in tumor cell killing may have safety advantages over immune checkpoint modulators. Unlike checkpoint modulators, neutrophil targeted therapies would not induce or require proliferation of immune cells, as circulating neutrophils are short-lived. In addition, the tumor-targeted antibody is eliminated when neutrophils kill the target tumor cell with the attached antibody, providing a negative feedback loop that diminishes immune stimulation as the therapeutic antibody is consumed by the target effector cells.

Another way that tumor-targeting therapeutic antibodies capable of engaging FPR-1 positive innate immune cells in tumor cell may prove useful is for treatment of cold tumors that have low mutational burden and therefore are not readily recognized by the immune system. Attracting and activating neutrophil-mediated tumor cell killing can result in local production of neoantigens in a cytokine rich environment such that cells of the adaptive immune system acquire the ability to recognize the tumor and target it for elimination.

A tumor targeted antibody capable of engaging neutrophils in tumor cell killing may also have advantages over toxic agent-based antibody drug conjugates (ADC) which are typically designed to release a toxic payload following internalization into the tumor cell. Like ADCs, a tumor targeted antibody capable of engaging neutrophils in tumor cell killing should recognize an antigen with high expression on tumor cells, with low expression on normal tissue. However, unlike ADCs, a tumor targeted antibody capable of engaging neutrophils in tumor cell killing requires agonist exposure to receptors on the surface of innate immune system, and thus is anticipated to function better with target antigens that have relatively less internalization potential.

While conjugated antibodies can be produced by reducing interchain disulfides to generate reactive thiols or utilizing surface lysines for conjugation, such conventional conjugation methods may consequently result in instability of the antibody or loss of binding affinity. Therefore, the present invention provides an antibody peptide conjugate with site specific addition(s) of N-formyl-methionine peptide-conjugates at engineered cysteine residues, which provide one or more of the following advantages (i) site specific addition allows a homogenous conjugation profile, which dictates the potency and maximal efficacy of the N-formyl-methionine peptide bioconjugate, (ii) a spacer can be used to retain the potency of the N-formyl-methionine peptide for migration and activation of human neutrophils when conjugated to the antibody, and increases the potency of the N-formyl-methionine peptide in vitro in human neutrophil migration assays, (iii) site specific addition retains the Fc-receptor interactions in IgG1 constructs, which can contribute to tumor cell killing, (iv) site specific addition allows the antibody to retain antigen binding affinity, which was achieved in some, but not all, prior literature examples, and (v) site specific conjugation maintains stability of the antibody which can be a significant advantage in the production of drug substance and stability of drug product.

The present invention also provides an IgG antibody, comprising engineered-cysteine residues for use in the generation of antibody conjugate compounds (also referred to as bioconjugates). More particularly, the present invention provides therapeutic compounds comprising tumor-targeting antibodies, comprised of engineered-cysteine residues, conjugated to a peptide or peptide mimetic capable of activating FPR-1 on cells of the innate immune system. In an embodiment, an antibody is conjugated to peptide or a peptide mimetic capable of agonizing FPR-1. In some particular embodiments, the peptide or peptide mimetic is a compound of one of the following formulas:

R—P₁—P₂—P₃—NH(CH₂CH₂O)ₙCH₂CH₂—Y Formula I.
wherein
R is a HC(═O)— or R¹NHC(═O)NH—;
R¹ is C₅-C₁₀ aryl which may be substituted or unsubstituted;
P₁ is Met or Nle;
P₂ is a peptide or peptide mimetic;
P₃ is Lysine with epsilon amino acylation;
n is an integer of from 6-24;
Y is maleimide, maleimide-diaminopropionic, iodoacetamide or vinyl sulfone;
or a salt thereof.

R—P₁—P₂—NH(CH₂CH₂O)ₙCH₂CH₂—P₃—Y Formula II.
wherein
R is a HC(═O)— or R¹NHC(═O)NH—;
R¹ is C₅-C₁₀ aryl which may be substituted or unsubstituted;
P₁ is Met or Nle;
P₂ is a peptide or peptide mimetic;
P₃ is Lysine with epsilon amino acylation;
n is an integer of from 6-24;
Y is maleimide, maleimide-diaminopropionic, iodoacetamide or vinyl sulfone;
or a salt thereof.

R-Met-X₁—X₂—X₃—X₄—NH(CH₂CH₂O)ₙCH₂CH₂—X₅—Y Formula III.
Wherein
R is a HC(═O)— or R¹NHC(═O)NH—;
R¹ is phenyl, 4-chlorophenyl, 4-methoxylphenyl, p-tolyl, m-tolyl, aryl, substituted aryl, or 2-allyl;
X₁ is Leu, Ile, Nle, diethylglycine, or dipropylglcyine;
X₂ is Phe, α-Me-Phe, DPhe, 4-F-Phe, 2-Nal, or 1-Nal;
X₃ is Glu, Leu, Nle, α-Me-Leu, DLeu, or absent;
X₄ is Glu, DGlu, γGlu, Gla, or absent;
X₅ is a C₂-C₁₀ diaminoakyl; and
Y is maleimide, maleimide-diaminopropionic, iodoacetamide or vinyl sulfone;
or a salt thereof.

In some other particular embodiments, the peptide is a compound of one of the following formulas:

[R—P₁—P₂—NH(CH₂CH₂O)ₙCH₂CH₂—]₂-Q-X-Y Formula IV.
wherein
R is a HC(═O)— or R¹NHC(═O)NH—;
R¹ is C₅-C₁₀ aryl which may be substituted or unsubstituted;
P₁ is Met or Nle;
P₂is a peptide or peptide mimetic;
n is an integer of from 6-24;
Q is an amino bifunctional residue that is capable of being acylated at an alpha amino group and at a side chain amino group;
X is a C₂-C₁₀ diaminoakyl; and
Y is maleimide, maleimide-diaminopropionic, iodoacetamide or vinyl sulfone;
or a salt thereof.

[[R—P₁—P₂—NH(CH₂CH₂O)ₙCH₂CH₂—]₄-(Q)₂-Q-X-Y Formula V.
wherein
R is a HC(═O)— or R¹NHC(═O)NH—;
R¹ is C₅-C₁₀ aryl which may be substituted or unsubstituted;
P₁ is Met or Nle;
P₂ is a peptide or peptide mimetic;
n is an integer of from 6-24;
Q is an amino bifunctional residue that is capable of being acylated at an alpha amino group and at a side chain amino group;
X is a C₂-C₁₀ diaminoakyl; and
Y is maleimide, maleimide-diaminopropionic, iodoacetamide or vinyl sulfone;
or a salt thereof.

[[[R—P₁—P₂—NH(CH₂CH₂O)ₙCH₂CH₂—]₈-(Q)₄-(Q)₂-Q-X-Y Formula VI.
wherein
R is a HC(═O)— or R¹NHC(═O)NH—;
R¹ is C₅-C₁₀ aryl which may be substituted or unsubstituted;
P₁ is Met or Nle;
P₂ is a peptide or peptide mimetic;
n is an integer of from 6-24;
Q is an amino bifunctional residue that is capable of being acylated at an alpha amino group and at a side chain amino group;
X is a C₂-C₁₀ diaminoakyl; and
Y is maleimide, maleimide-diaminopropionic, iodoacetamide or vinyl sulfone;
or a salt thereof.

The compounds of Formulas IV-VI comprise two or more chemoattractants linked together via an amino bifunctional residue (represented by “Q”). In some embodiments, Q is Lys, Orn, Dap, or Dab. In a preferred embodiment, the bifunctional residue is a lysine or ornithine residue. The bifunctional residue can be linked to two additional amino bifunctional residues through each amino group, thereby increasing the number of chemoattractants to four chemoattractants. Additional bifunctional residues allow for additional numbers of chemoattractants. In a preferred embodiment, the number of chemoattractants is no more than eight. For example, if Q₂ is a repetition of a lysine-branched residue, the structure is the following:

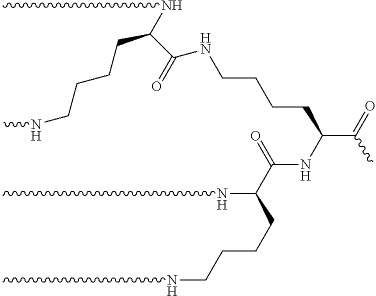

The present invention provides the compound of any one of Formulas I-VI, wherein P2 is given by X₁-X₂-X₃-X₄, and
X₁ is Leu, Ile, Nle, diethylglycine, or dipropylglcyine;
X₂ is Phe, α-Me-Phe, DPhe, 4-F-Phe, 2-Nal, or 1-Nal;
X₃ is Glu, Leu, Nle, α-Me-Leu, DLeu, or absent; and
X₄ is Glu, DGlu, γGlu, Gla, or absent.

In some embodiments, the compound of any one of Formulas I, II, III, IV, V or VI is capable of agonizing formyl peptide receptor 1 and forming a covalent linkage with a protein. In some embodiments, the compound of any one of Formulas I, II, III, IV, V, or VI is conjugated to an antibody via a linker. In some particular embodiments, the compound is conjugated via a maleimide-PEG linker as described herein. In some particular embodiments, the PEG linker is bound to the diaminoalkyl of X. In some particular embodiments, the PEG linker is absent and the compound of any one of Formulas I, II, III, IV, V, or VI is bound directly to the diaminoalkyl of X. In some such embodiments, the compounds derived from any one of Formulas I, II, III, IV, V, or VI are capable of activating formyl peptide receptors on the surface of innate immune cells, such as neutrophils.

The embodiment of the current invention is also useful in a non-tumor context for engaging innate immune cells in specific elimination of the target cells of interest that have utility beyond cancer therapy. In situations where elimination of normal cells is desirable, for example in hypertrophic tissues, tissues with restricted access, or viral infected cells, an antibody that specifically targets the cells of interest that is also capable of activating cells of the innate immune system to provided targeted cell killing would be useful for eliminating those target tissues or infected cells.

The present invention contemplates a range of linkers to attach FPR-1 agonists to the engineered cysteine residues (Yao et al., Int J Mol Sci. 2016 Feb. 2; 17(2). pii: E194. doi: 10.3390/ijms17020194). Examples provided include maleimide-based linkers to form a thioether linkage to the cysteines, The use of another linker, such as a haloacetyl linker, may also be used to conjugate the antibody.

Thus, the present invention provides an antibody comprising an IgG heavy chain and light chain constant region wherein said constant region comprises at least one cysteine. In an embodiment, the constant region comprises an unpaired free cysteine on the surface. In another embodiment, the constant region comprises an engineered cysteine. In some particular embodiments, the constant region comprises at least one engineered cysteine at one of the following residues: residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain.

The present invention also provides an antibody comprising an IgG heavy chain constant region wherein said constant region comprises a cysteine at residue 124 in the C_{H}1 domain, and a cysteine at one, but not all, of residue 157 and 162 in the C_{H}1 domain and residues 375 and 378 in the C_{H}3 domain. As a particular embodiment, the IgG heavy chain constant region is a human, mouse, rat or rabbit IgG constant region. Even more particular, the IgG heavy chain constant region is a human IgG1, human IgG2, or human IgG4 isotype, and even more particularly, human IgG1 or human IgG4. As an even more particular embodiment the IgG heavy chain constant region is a human IgG1 isotype and given by the amino acid sequence of SEQ ID NO: 17, 18, 19 or 52 and even more particularly, the amino acid sequence of SEQ ID NO: 20,21 or 53. As an even further particular embodiment to the afore-mentioned antibodies comprising human IgG1 heavy chain constant regions, said constant regions further comprise an isoleucine substituted at residue 247 and a glutamine substituted at residue 339. In another embodiment, the constant regions comprise an isoleucine substituted at residue 247, a glutamine substituted at residue 339, and a glutamic acid substituted at residue 332. As an alternative particular embodiment, the IgG heavy chain constant region is a human IgG4 isotype and given by the amino acid sequence of SEQ ID NO: 12, 13, 14, 54 or 55 and even more particularly, the amino acid sequence of SEQ ID NO: 15, 16, 56 or 57. As an even further particular embodiment to the afore-mentioned antibodies comprising human IgG4 heavy chain constant regions, said constant regions further comprise a proline substituted at residue 228, an alanine substituted at residue 234, and an alanine substituted at residue 235.

The present invention further provides an antibody comprising two heavy chain IgG constant regions wherein each IgG constant region comprises at least one cysteine. In an embodiment, each IgG constant region comprises a cysteine at one of the following residues: residue 124 in the C_{H}1 domain, residue 157 in the C_{H}1 domain, residue 162 in the C_{H}1 domain, residue 375 in the C_{H}3 domain, and residue 378 in the C_{H}3 domain. The present invention also provides any of the afore-mentioned antibodies comprising two heavy chain IgG constant regions wherein each IgG constant region comprises a cysteine at residue 124 in the C_{H}1 domain, and a cysteine at one, but not all, of residue 157 and 162 in the C_{H}1 domain and residues 375 and 378 in the C_{H}3 domain of each heavy chain. More particularly, each IgG constant region is human, mouse, rat or rabbit IgG, and even more particularly human IgG1, human IgG2, or human IgG4 isotype, and even more particularly, human IgG1 or human IgG4. As an even more particular embodiment each IgG heavy chain constant region is a human IgG1 isotype and is given by the amino acid sequence of SEQ ID NO: 17, 18,19 or 52 and even more particularly, the amino acid sequence of SEQ ID NO: 20,21 or 53. As an even further particular embodiment to the afore-mentioned antibodies comprising two human IgG1 heavy chain constant regions, said constant regions further comprise an isoleucine substituted at residue 247 and a glutamine substituted at residue 339. In another emdobiment, the constant regions comprise an isoleucine substituted at residue 247, a glutamine substituted at residue 339, and a glutamic acid substituted at residue 332. As an alternative particular embodiment, each IgG heavy chain constant region is a human IgG4 isotype and is given by the amino acid sequence of SEQ ID NO: 12, 13, 14, 54 or 55 and even more particularly, the amino acid sequence of SEQ ID NO: 15,16, 56 or 57. As an even further particular embodiment to the afore-mentioned antibodies comprising two human IgG4 heavy chain constant regions, said constant regions further comprise a proline substituted at residue 228, an alanine substituted at residue 234, and an alanine substituted at residue 235.

The present invention further provides any of the afore-mentioned antibodies wherein each cysteine at residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain is conjugated to a chemoattractant. In an embodiment, the chemoattractant is an f-Met peptide, small molecule FPR-1 agonist, PRR agonist, peptide mimetics, N-ureido-peptide, or bacterial sugar. In a particular embodiment, the chemoattractant is an N-formyl-methionine peptide. In some embodiments, the chemoattractant is conjugated to the antibody cysteine via a maleimide-linker, wherein said linker forms a covalent attachment to said IgG heavy chain and light chain constant regions through a thioether bond between a maleimide functional group and the cysteine (located at residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain.) and also forms a covalent attachment to said N-formyl-methionine peptide through an amide bond to the epsilon amino side chain of the C-terminal lysine of said N-formyl-methionine peptide. In an embodiment, the present invention provides any of the afore-mentioned antibodies wherein each cysteine referred to herein is conjugated to an N-formyl-methionine peptide via a maleimide-linker, wherein said linker forms a covalent attachment to said IgG heavy chain constant regions through a thioether bond between a maleimide functional group and the cysteine, and also forms a covalent attachment to said N-formyl-methionine peptide through an amide bond to the epsilon amino side chain of the C-terminal lysine of said N-formyl-methionine peptide. As a particular embodiment, the present invention further provides an antibody compound comprising two heavy chain IgG constant regions wherein each IgG constant region comprises a cysteine at residue 124 in the C_{H}1 domain, and a cysteine at one, but not all, of residues 157 and 162 in the C_{H}1 domain and 375 and 378 in the C_{H}3 domain, wherein each cysteine at residue 124 of each C_{H}1 domain, and each cysteine at residue 157 or 162 in the C_{H}1 domain, 375 or 378 of each C_{H}3 domain is conjugated to an N-formyl-methionine peptide via a maleimide linker, wherein said linker is covalently attached to said antibody through a thioether bond between a maleimide functional group and the cysteine at residue 124, 157 or 162 and 375 or 378 of each IgG constant region, and to said N-formyl-methionine peptide through an amide bond to the epsilon amino side chain of the C-terminal lysine of said N-formyl-methionine peptide. More particular to the afore-mentioned conjugated antibodies, the maleimide linker has the formula

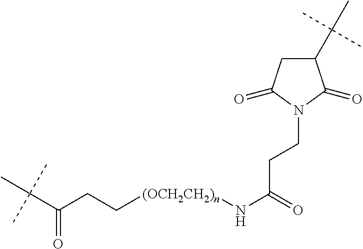

wherein n=1-24, more particular n=6-24, and even more particular n=12. Even more particular, the N-formyl-methionine peptide is N-formyl-methionine-leucine-phenylalanine-X (SEQ ID NO: 22), wherein X is lysine modified by amide bond formation to the maleimide linker. More particular still, each IgG constant region of said conjugated antibody compound is human IgG1 or human IgG4 isotype, and even more particularly, each IgG heavy chain constant region is a human IgG1 isotype and further comprises an isoleucine substituted at residue 247 and a glutamine substituted at residue 339, or each IgG heavy chain constant region is a human IgG4 isotype and further comprises a proline substituted at residue 228, an alanine substituted at residue 234, and an alanine substituted at residue 235.

The engineered-cysteine residues of the present invention may be incorporated into IgG constant regions of existing cancer therapeutic antibodies to facilitate generation of alternative N-formyl-methionine peptide-conjugated immunotherapeutics. Alternatively, the heavy chain CDRs or variable domains of existing cancer therapeutic antibodies may be combined with IgG constant regions containing the engineered-cysteine residues of the present invention to generate conjugated immunotherapeutics. Exemplary cancer therapeutics for these applications include IgG1 therapeutic antibodies targeting solid tumors, including tumors expressing HER-2 (i.e, IgG1 antibodies such as trastuzumab and pertuzumab), liquid tumors, including liquid tumors expressing CD20 (i.e., IgG1 and IgG1-enhanced ADCC antibodies such as rituximab, ofatumumab, obinutuzumab, and AME133v) and antibodies targeting c-Met-expressing tumors (i.e., emibetuzumab).

The N-formyl methionine peptide-conjugated antibodies as disclosed herein may also serve as a platform to further conjugate cytotoxic agents to achieve greater efficacy, or as an alternative to the drug conjugate in antibody drug conjugates that target antigens overexpressed in cancer cells. Target antigens with exemplary antibody drug conjugates include, but are not limited to, GPNMB (glembatumumab vedotin), CD56 (lorvotuzumab mertansine (IMGN-901)), TACSTD2 (TROP2; sacituzumab govitecan, (IMMU-132)), CEACAM5 (labetuzumab SN-38), folate receptor-α (mirvetuximab soravtansine (IMGN-853), vintafolide), mucin 1 (sialoglycotope CA6; SAR-566658) STEAP1 (vandortuzumab vedotin (RG-7450)), mesothelin (DMOT4039A, anetumab ravtensine (BAY-94-9343), BMS-986148), nectin 4 (enfortumab vedotin (ASG-22M6E); ASC-22CE), ENPP3 (AGS-16M8F), guanylyl cyclase C (indusatumab vedotin (MLN-0264)), SLC44A4 (ASG-5ME), NaPi2b, (lifastuzumab vedotin), CD70 (TNFSF7; DNIB0600A, AMG-172, MDX-1243, vorsetuzumab mafodotin (SGN-75)) CA9 carbonic anhydrase (BAY79-4620), 5T4 (TPBG; PF 06263507) SLTRK6 (ASG-15ME), SC-16 (anti-Fyn3; SC16LD6.5), tissue factor (HuMax-TF-ADC (TF-011-MMAE)), LIV-1 (ZIP6; SGN-LIV1A), P-Cadherin (PCA062) PSMA (MLN2704, PSMA-ADC), Fibronectin Extra-domain B (Human mAb L19 and F8), endothelin receptor ETB (RG-7636), VEGFR2 (CD309; anti-VEGFR-2ScFv-As203-stealth nanoparticles), Tenascin c (anti-TnC-A1 antibody SIP (F16)), periostin (anti-periostin antibody), DLL3 (rovalpituzumab soravtansine), HER 2 (T-DM1, ARX788, SYD985), EGFR (ABT-414, IMGN289 AMG-595), CD30 (brentuximab vedotin, iratumumab MDX-060), CD22 (Inotuzumab ozogamicin (CMC-544), pinatuzumab vedotin, epratuzumab SN38), CD79b (polatuzumab vedotin), CD19 (coltuximab ravtansine, SAR-3419, SGN-CD19A), CD138 (indatuximab ravtansine), CD74 (milatuzumab doxorubicin), CD37 (IMGN-529), CD33 (gemtuzumab ozogamicin, IMGN779, SGN CD33 A,) and CD98 (IGN523). (see e.g., Thomas et al, Lancet Oncol. 2016 June; 17(6)e254-62 and Diamantis and Banerji, Brit. Journ. Cancer, 2016; 114, 362-367).

Thus, the present invention further provides an IgG antibody comprising the heavy chain and light chain CDRs of any of the afore-mentioned cancer therapeutic antibodies, wherein each IgG constant region comprises a cysteine at residue 124 in the C_{H}1 domain, and a cysteine at one, but not all, of residue residue 157 and 162 in the C_{H}1 domain and 375 and 378 in the C_{H}3 domain. Further, the present invention provides any of the afore-mentioned cysteine-engineered antibodies wherein each cysteine at residue 124 of each IgG constant region, and each cysteine at residue 157, 162, 375 or 378 of each IgG constant region is conjugated to an N-formyl-methionine peptide via a maleimide-PEG linker, all as described herein.

The present invention provides a compound that is an antibody containing at least one cysteine conjugated to a chemoattractant, optionally through a linker, that is capable of attracting and/or activating one or more cells of the immune system, and wherein the agent is conjugated to the antibody at one or more cysteine residues within the antibody. In some embodiments, the antibody comprises an IgG heavy chain constant region, wherein said constant region comprises a cysteine at at least one of the following residues: residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain. In some embodiments, the cysteine is an engineered cysteine. In further embodiments, the number of engineered cysteines on each heavy chain and/or light chain is between one and three. In other embodiments, the antibody is conjugated to the chemoattractant through a linker. In some embodiments, the linker is a maleimide-PEG linker or a Mal-Dap linker. In other embodiments, the chemoattractant is a f-Met peptide, small molecule FPR-1 agonists, PRR agonist, peptide mimetics, N-ureido-peptide, or bacterial sugar.

The present invention provides a compound that is an antibody containing at least one cysteine conjugated to a chemoattractant, optionally through a linker, that is capable of attracting and/or activating one or more cells of the immune system, and wherein the agent is conjugated to the antibody at one or more cysteine residues within the antibody, and wherein the chemoattractant is the compound of any one of Formula I, Formula II, Formula III, Formula IV, Formula V, or Formula VI, as described herein. In some embodiments, the compound is capable of attracting and activating one or more cells of the immune system. In some particular embodiments, the compound is capable of attracting and activating one or more cells of the innate immune system. In a preferred embodiment, a linker is present.

In addition, the present invention also provides any of the antibodies, IgG heavy chain constant regions, and N-formyl methionine peptide-conjugates thereof, each as specifically exemplified herein. As a further embodiment, the present invention provides any of the antibodies, IgG heavy chain constant regions, conjugated antibodies, or a nucleic acids encoding one of the same, in “isolated” form. As used herein, the term “isolated” refers to a protein, polypeptide, or nucleic acid which is free or substantially free from other macromolecular species found in a cellular environment.

The present invention further provides pharmaceutical compositions comprising any of the N-formyl methionine peptide-conjugated antibodies as described herein and a pharmaceutically acceptable carrier or excipient. In addition, the present invention further provides a method of treating solid cancers, including breast, lung, prostate, skin, colorectal, bladder, kidney, liver, thyroid, endometrial, muscle, bone mesothelial, vascular and fibrous cancers and associated metastases, and liquid tumors, including leukemias and lymphomas, comprising administering to a patient in need thereof an effective amount of an N-formyl-methionine peptide-conjugated antibody, or a pharmaceutical composition thereof, each as described herein. Further, the present invention further provides any of the N-formyl-methionine peptide-conjugated antibodies as described herein, and the pharmaceutical compositions thereof, for use in therapy. In particular, the present invention provides any of the N-formyl-methionine peptide-conjugated antibodies as described herein, and the pharmaceutical compositions thereof, for use in the treatment of breast cancer, lung cancer, prostate cancer, skin cancer, colorectal cancer, bladder cancer, kidney cancer, liver cancer, thyroid cancer, endometrial cancer, muscle cancer, bone mesothelial cancer, vascular and fibrous cancers, leukemia and lymphoma. As a particular embodiment to the methods, uses and compositions herein, the N-formylated methionine peptide is N-formyl-Met-Leu-Phe-Lys-OH.

### Definitions

The general structure of an “IgG antibody” is very well-known. A wild type (WT) antibody of the IgG type is hetero-tetramer of four polypeptide chains (two identical “heavy” chains and two identical “light” chains) that are cross-linked via intra-and inter-chain disulfide bonds. Each heavy chain (HC) is comprised of an N-terminal heavy chain variable region (“V_{H}”) and a heavy chain constant region. The heavy chain constant region is comprised of three domains (C_{H}1, C_{H}2, and C_{H}3) as well as a hinge region (“hinge”) between the C_{H}1 and C_{H}2 domains. Each light chain (LC) is comprised of an N-terminal light chain variable region (“V_{L}”) and a light chain constant region (“C_{L}”). The V_{L} and C_{L} regions may be of the kappa (“κ”) or lambda (“λ”) isotypes (“C_{κ}” or “C_{λ}”, respectively). Each heavy chain associates with one light chain via interfaces between the heavy chain and light chain variable domains (the V_{H}/V_{L} interface) and the heavy chain constant C_{H}1 and light chain constant domains (the C_{H}1/C_{L} interface). The association between each of the V_{H}-C_{H}1 and V_{L}-C_{L} segments forms two identical antigen binding fragments (Fabs) which direct antibody binding to the same antigen target or epitope. Each heavy chain associates with the other heavy chain via interfaces between the hinge-C_{H}2-C_{H}3 segments of each heavy chain, with the association between the two C_{H}2-C_{H}3 segments forming the Fc region of the antibody. Together, each Fab and the Fc form the characteristic “Y-shaped” architecture of IgG antibodies, with each Fab representing the “arms” of the “Y.” IgG antibodies can be further divided into subtypes, e.g., IgG1, IgG2, IgG3, and IgG4 which differ by the length of the hinge regions, the number and location of inter-and intra-chain disulfide bonds and the amino acid sequences of the respective HC constant regions.

The variable regions of each heavy chain-light chain pair associate to form binding sites. The heavy chain variable region (V_{H}) and the light chain variable region (V_{L}) can be subdivided into regions of hypervariability, termed complementarity determining regions (“CDRs”), interspersed with regions that are more conserved, termed framework regions (“FR”). Each V_{H} and V_{L} is composed of three CDRs and four FRs, arranged from amino-terminus to carboxy-terminus in the following order: FR1, CDR1, FR2, CDR2, FR3, CDR3, FR4. CDRs of the heavy chain may be referred to as “CDRH1, CDRH2, and CDRH3” and the 3 CDRs of the light chain may be referred to as “CDRL1, CDRL2 and CDRL3.” The FRs of the heavy chain may be referred to as HFR1, HFR2, HFR3 and HFR4 whereas the FRs of the light chain may be referred to as LFR1, LFR2, LFR3 and LFR4. The CDRs contain most of the residues which form specific interactions with the antigen.

The compounds and methods of the present invention comprise designed amino acid modifications at particular residues within the constant regions of heavy chain polypeptides. As one of ordinary skill in the art will appreciate, various numbering conventions may be employed for designating particular amino acid residues within IgG constant and variable region sequences. Commonly used numbering conventions include the “Kabat Numbering” and “EU Index Numbering” systems. “Kabat Numbering” or “Kabat Numbering system”, as used herein, refers to the numbering system devised and set forth by the authors in Kabat et al., Sequences of Proteins of Immunological Interest, 5th Ed, Public Health Service, National Institutes of Health, Bethesda, MD (1991) for designating amino acid residues in both variable and constant domains of antibody heavy chains and light chains. “EU Index Numbering” or “EU Index Numbering system”, as used herein, refers to the numbering convention for designating amino acid residues in antibody heavy chain constant domains, and is also set forth in Kabat et al (1991). Other conventions that include corrections or alternate numbering systems for variable domains include Chothia (Chothia C, Lesk AM (1987), J Mol Biol 196:901-917; Chothia, et al. (1989), Nature 342:877-883), IMGT (Lefranc, et al. (2003), Dev Comp Immunol 27:55-77), and AHo (Honegger A, Pluckthun A (2001) J Mol Biol 309:657-670). Unless otherwise expressly stated herein, all references to immunoglobulin heavy chain constant region C_{H}1, hinge, C_{H}2, and C_{H}3 amino acid residues (i.e. numbers) appearing in the specification, Examples and Claims are based on the EU Index Numbering system. With knowledge of the residue number according to EU Index Numbering, one of ordinary skill can apply the teachings of the art to identify amino acid sequence modifications within the present invention, according to any commonly used numbering convention. Note, while the specification, Examples and Claims of the present invention employ EU Index Numbering to identify particular amino acid residues, it is understood that the SEQ ID NOs appearing in the Examples and Sequence Listing accompanying the present application, as generated by Patent In Version 3.5, provide sequential numbering of amino acids within a given polypeptide and, thus, do not conform to the corresponding amino acid residue numbers as provided by EU Index Numbering.

The polypeptide chains described herein are depicted by their sequence of amino acids from N-terminus to C-terminus, when read from left to right, with each amino acid represented by either their single letter or three-letter amino acid abbreviation. Unless otherwise stated herein, all amino acids used in the preparation of the polypeptides of the present invention are L-amino acids. The “N-terminus” (or amino terminus) of an amino acid, or a polypeptide chain, refers to the free amine group on the amino acid, or the free amine group on the first amino acid residue of the polypeptide chain. Further, the term “N-terminal amino acid” refers to the first amino acid in a polypeptide chain. Likewise, the “C-terminus” (or carboxy terminus) of an amino acid, or a polypeptide chain, refers to the free carboxy group on the amino acid, or the free carboxy group on the final amino acid residue of the polypeptide chain. Further, the term “C-terminal amino acid” refers to the last amino acid in a polypeptide chain.

As used herein, the phrase “ . . . a/an [amino acid name] substituted at residue . . . ”, in reference to a heavy chain or light chain polypeptide, refers to substitution of the parental amino acid with the indicated amino acid. By way of example, a heavy chain comprising “an alanine substituted at residue 235” refers to a heavy chain wherein the parental amino acid sequence has been mutated to contain an alanine at residue number 235 in place of the parental amino acid. Such mutations may also be represented by denoting a particular amino acid residue number, preceded by the parental amino acid and followed by the replacement amino acid. For example, “F235A” refers to a replacement of a phenylalanine at residue 235 with an alanine. Similarly, “235A” refers to replacement of a parental amino acid with an alanine. An “engineered” cysteine refers to substitution of the parental amino acid with a cysteine.

As used herein, “N-formyl-methionine peptide” refers to a peptide of 4-10 amino acids in length, wherein the N-terminal amino acid is a formylated methionine and the C-terminal amino acid is a lysine. A particular N-formyl-methionine peptide is the peptide N-formyl-methionine-leucine-phenylalanine-lysine-OH (“fMLFK;” SEQ ID NO: 23).

As used herein, “linker” refers to a structure that connects two or more additional structures. Examples of linkers include peptide linkers, protein linkers, and PEG linkers. A “maleimide-PEG linker”, as used herein, refers to a chemical moiety comprising a polyethylene glycol (PEG) polymer of the formula “—(O—CH₂—CH₂)ₙ—”, wherein “n” is 6-24, and a derivatized maleimide functional group, wherein said linker forms a covalent attachment to an IgG antibody heavy chain through a thioether bond between a maleimide functional group and a cysteine residue in the heavy chain constant region, and also forms a covalent attachment to an N-formyl-methionine peptide through an amide bond to the epsilon amino side chain of the C-terminal lysine of said N-formyl-methionine peptide. As a particular embodiment, the maleimide-PEG linker of the compounds of the present invention has the following structure, wherein the dashed lines represent the locations of covalent attachments to the IgG antibody heavy chain and the N-formyl-methionine peptide:

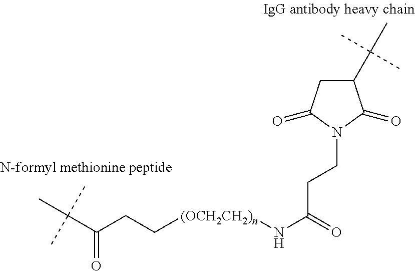

wherein, “n”=6-24 and more particularly, “n”=12.

In the present case, the reagent used to prepare the test compounds employed in the Examples below (Mal-dPEG12-OH (QuantaBiodesign Cat #10285, Lot IH1-A1240-80)) is a monodisperse regent, meaning it contains a discrete number of ethyl-oxy monomer (O—CH₂—CH₂) units. Likewise, using this reagent will produce conjugated antibody compounds which contain maleimide-PEGₙ linkers having n=12 (O—CH₂—CH₂) units.

However, as one of skill in the art will appreciate, pegylation reagents are often described by reference to the molecular weight (in daltons or kilodaltons) of the PEG polymer portion of the PEG-containing compounds in the reagent. Further, many commercially available PEG-containing reagents generally have some degree of polydisperity, meaning that the number of repeating ethylene glycol monomer units contained within the reagent (the “n”) varies over a range, typically over a narrow range. Thus, the reference to the PEG polymer molecular weight in a polydisperse reagent is typically a reference to the average molecular weight of the PEG polymers contained within the reagent. The ethyl-oxy monomer (O—CH₂—CH₂) of the reagent used to prepare the conjugated antibody compounds of the present invention has a molecular weight of about 44 g/mol or 44 daltons. Thus, one of skill in the art can readily determine the value of “n” when using a polydisperse pegylation reagent denoted by its average molecular weight and, likewise, the value of “n” in a resulting conjugated antibody compound.

The term “substituted” as used in the phrase “R1 is C₅-C₁₀ aryl which may be substituted or unsubstituted,” for example, herein signifies that one or more substituents may be present, said substituents being selected from atoms and groups which, when present in the compound of Formula II, Formula III, Formula IV, Formula V or Formula VI, do not prevent the compound from functioning as a chemoattractant. Examples of substituents which may be present in a substituted C₅-C₁₀ aryl include Hydroxyls, Halides (I, Cl, F, Br), Alkoxy groups (MeO—, EtO—, PrO or C₁-C₄), or Alkyl groups (Me-, Et-, Pr or C₁-C₄) that are covalently linked to the aryl structure.

The term diaminoalkyl is given by the structure —NH(CH₂)ₙNH—, wherein n=2-10.

A formyl group consists of a carbonyl bonded to hydrogen and is given by the following structure: CH(═O), or

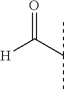

Maleimide-diaminopropionic acid is coupled to Y via amide bond to a free amine, and refers to the structure:

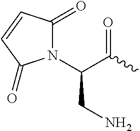

Maleimide is coupled to Y via amide bond to a free amine, and refers to 3-maleimidopropionic acid, given by the following structure:

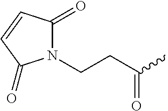

As used herein, the term “patient in need thereof” refers to a human or non-human mammal, and more preferably a human, which has been diagnosed as having a condition or disorder for which treatment or administration with a compound of the present invention is indicated.

As used herein the term “effective amount” refers to the amount or dose of a conjugated antibody compound of the present invention, which upon single or multiple dose administration to the patient, provides the desired pharmacological effect in the patient. An effective amount can be readily determined by the attending diagnostician, as one skilled in the art, by considering a number of factors such as the species of mammal; its size, age, and general health; the specific disease or surgical procedure involved; the degree or severity of the disease or malady; the response of the individual patient; the particular compound or composition administered; the mode of administration; the bioavailability characteristics of the preparation administered; the dose regimen selected; and the use of any concomitant medications.

The cysteine-engineered IgG antibodies for use in the present invention can be produced using techniques well known in the art, such as recombinant expression in mammalian or yeast cells. In particular, the methods and procedures of the Examples herein may be readily employed. In addition, the IgG antibodies of the present invention may be further engineered to comprise framework regions derived from fully human frameworks. A variety of different human framework sequences may be used in carrying out embodiments of the present invention. As a particular embodiment, the framework regions employed in the IgG antibodies of the present invention are of human origin or are substantially human (at least 95%, 97% or 99% of human origin.) The sequences of framework regions of human origin are known in the art and may be obtained from The Immunoglobulin Factsbook, by Marie-Paule Lefranc, Gerard Lefranc, Academic Press 2001, ISBN 012441351.

Expression vectors capable of directing expression of genes to which they are operably linked are well known in the art. Expression vectors contain appropriate control sequences such as promoter sequences and replication initiation sites. They may also encode suitable selection markers as well as signal peptides that facilitate secretion of the desired polypeptide product(s) from a host cell. The signal peptide can be an immunoglobulin signal peptide or a heterologous signal peptide. Nucleic acids encoding desired polypeptides, for example the HC and LC components of the conjugated IgG antibodies of the present invention, may be expressed independently using different promoters to which they are operably linked in a single vector or, alternatively, the nucleic acids encoding the desired products may be expressed independently using different promoters to which they are operably linked in separate vectors. Single expression vectors encoding both the HC and LC components of the cysteine-engineered IgG antibodies of the present invention may be prepared using standard methods.

As used herein, a “host cell” refers to a cell that is stably or transiently transfected, transformed, transduced or infected with nucleotide sequences encoding a desired polypeptide product or products. Creation and isolation of host cell lines producing an IgG antibody for use in the present invention can be accomplished using standard techniques known in the art. Mammalian cells are preferred host cells for expression of the cysteine-engineered IgG antibodies according to the present invention. Particular mammalian cells include HEK293, NS0, DG-44, and CHO cells. Preferably, assembled proteins are secreted into the medium in which the host cells are cultured, from which the proteins can be recovered and isolated. Medium into which a protein has been secreted may be purified by conventional techniques. For example, the medium may be applied to and eluted from a Protein A or G column using conventional methods. Soluble aggregate and multimers may be effectively removed by common techniques, including size exclusion, hydrophobic interaction, ion exchange, hydroxyapatite or mixed modal chromatography. Recovered products may be immediately frozen, for example at −70° C., or may be lyophilized. As one of skill in the art will appreciate, when expressed in certain biological systems, e.g. mammalian cell lines, antibodies are glycosylated in the Fc region unless mutations are introduced in the Fc to reduce glycosylation. In addition, antibodies may be glycosylated at other positions as well.

As used herein, a “bacterial sugar” refers to a polysaccharide at the outer surface of a bacteria. An example of a bacterial sugar is carrageenan.

As used herein, a “mimetic” refers to a molecule that functions similar to a naturally-occurring molecule. For example, a peptide mimetic can be a molecule such as a peptide, a modified peptide, or any other molecule that biologically mimics active ligands of hormones, cytokines, enzyme substrates, viruses or other naturally-occurring molecules.

As used herein, a “chemoattractant” refers to a structure, such as a peptide, that is capable of attracting and/or activating cells of the immune system. In a preferred embodiment, a chemoattractant is a structure that is capable of attracting and activating cells of the immune system. Examples of a chemoattractant include f-Met peptide, small molecule FPR-1 agonists, PRR agonist, peptide mimetics, N-ureido-peptide, and bacterial sugar. More specific examples include the compound of any one of Formulas I-IV, and the peptides of any one of SEQ ID NOs 22, 36-39.

The following Examples further illustrate the invention and provide typical methods and procedures for carrying out various particular embodiments of the present invention. However, it is understood that the Examples are set forth the by way of illustration and not limitation, and that various modifications may be made by one of ordinary skill in the art.

### EXAMPLE 1

### Design of IgG Heavy Chain Constant Regions Containing Engineered-Cysteine Residues

IgG heavy chain constant region residues are selected for mutation to allow the use of the engineered cysteine designs with parental mAbs having diverse variable or antigen-binding domains. Briefly, valine, alanine, and serine residues in the constant domains which are not critical for the antibody secondary and tertiary structure are selected for initial mutation in silico. Using the published crystal structures of a C_{H}1-CKappa Fab (pdb: 4DTG) and IgG4 Fc (pdb: 4C55), multiple different antibody single cysteine-engineered constructs are designed. Genes encoding each mutant design are constructed in human IgG4 heavy chain and kappa light chain plasmids and expressed in cells and the unconjugated engineered-cysteine containing mAbs are characterized by expression level and analytical profile. Constructs which retain essentially the same target binding affinity and expression level as the parental wild type mAb (as determined by ELISA), with minimal high molecular weight aggregates prior to conjugation (<10%), are scaled up and further characterized.

More than twenty mAb constructs with single cysteine mutations engineered into each HC and LC constant domains are then expressed in HEK293 cells, purified and conjugated via a linker to a cytotoxic payload such as monomethyl auristatin E (MMAE) and cryptophycin. Conjugation efficiency is monitored by standard procedures such as ESI-TOF mass spectrometry or Hydrophobicity Index Chromatography (HIC) while aggregation propensity is measured by analytical size exclusion chromatography. Constructs with greater than ˜60% conjugation efficiency and less than ˜10% high molecular aggregates after conjugation to both payloads are further examined for ex vivo plasma and in vivo stability studies.

Briefly, conjugate is incubated with plasma for several days and analyzed by mass spectrometry to confirm that the payload is still conjugated on the antibody. Conjugated constructs containing residue mutations at S124C, S157C, A162C, S375C, or A378C in each HC are found to have suitable stability. The HC 124C mutation can be combined with either 157C, 162C, 375C or 378C to yield higher antibody-drug ratio. Furthermore, additional single cysteine enginnered emibetuzmab mutants in heavy chain residue 124, 157 and 162 in the C_{H}1 domain, residue 262 in the C_{H}2 domain and residue 375, 378 and 397 in the C_{H}3 domain, and light chain residue 156, 171, 191, 193, 202 and 208 in the Cₖₐₚₚₐ domain were generated for conjugation with various formyl peptides.

In addition to monovalent IgG antibodies including engineered cysteines with conjugated chemoattractants, bivalent antibody contructs can also be developed with engineered cysteines having conjugated chemoattractants as disclosed herein. Bivalent antibody constructs with engineered cysteines include, but are not limited to, an IgG-scFv format (as reported in PCT/US2015/058719) and bivalent IgG formats (as disclosed in US 2018/0009908). According to such bivalent antibody contructs, site specific engineered cysteines include surface exposed cysteines for conjugation of chemoattractant to the bispecific antibody. According to a specific embodiment (bispecific antibody having a bivalent IgG format with two HCs of SEQ ID NO: 34, 35 and two LCs of SEQ ID NO: 58, 59), cysteines at heavy chain residue 124 and 378 are engineered for conjugation of chemoattractant. Expression and assembly of such exemplified ambodiment was unaltered, while conjugation with test peptides delivered comparable CR to monospecific antibodies.

### EXAMPLE 2

### Synthesis of Pegylated fMLFK Peptides

### Example 2 (A): Synthesis of formyl-Met-Leu-Phe-Lys (Mal-PEG12)-OH (“Peptide-'183”) (SEQ ID NO:22).

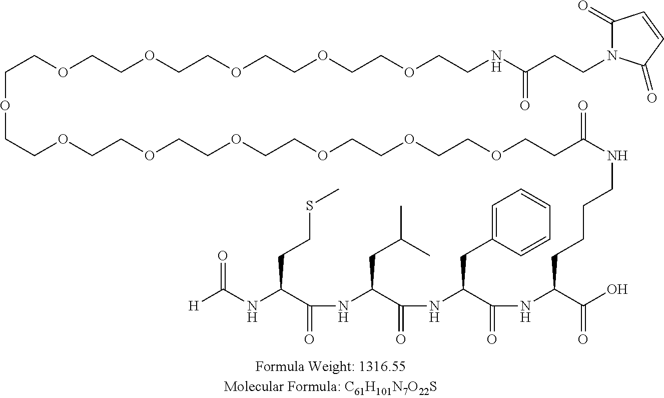 [SMILES: CSCC[C@H](NC=O)C(=O)N[C@@H](CC(C)C)C(=O)N[C@@H](Cc1ccccc1)C(=O)N[C@@H](CCCCNC(=O)CCOCCOCCOCCOCCOCCOCCOCCOCCOCCOCCOCCOCCNC(=O)CCN1C(=O)C=CC1=O)C(=O)O]

Peptide-'183 with hydrolyzed maleimido group used as unconjugated peptide.

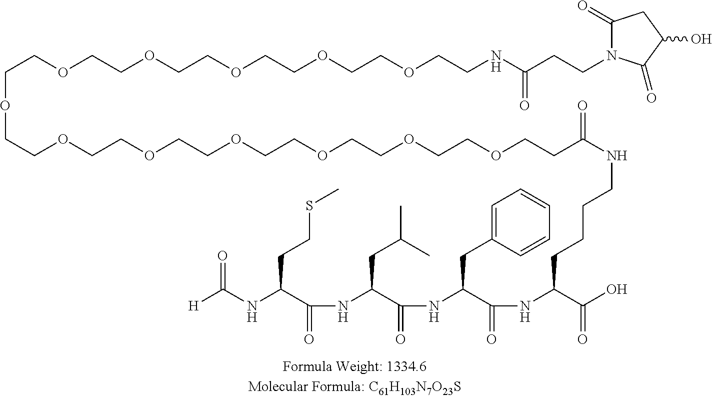 [SMILES: CSCC[C@H](NC=O)C(=O)N[C@@H](CC(C)C)C(=O)N[C@@H](Cc1ccccc1)C(=O)N[C@@H](CCCCNC(=O)CCOCCOCCOCCOCCOCCOCCOCCOCCOCCOCCOCCOCCNC(=O)CCN1C(=O)CC(O)C1=O)C(=O)O]

The chemotactic peptide formyl-Met-Leu-Phe-Lys-OH (SEQ ID NO:23) is synthesized and purified as the HCl salt. The material is used as a substrate for further derivatization at the ε-amino group of the lysine.

The peptide is produced via manual solid phase peptide synthesis using standard Fmoc/tBu chemistry at a 0.3 mmol scale in a 100 mL fritted glass manual reaction vessel from Ace Glassware Inc. The solid support used for the synthesis was Fmoc-Lys (Boc)-Wang resin, (NovaBiochem, Cat #8.56013, Lot S6696713-529), 100-200 mesh, with a substitution of 0.57 meq/g. Standard amino acids used were: Fmoc-Phe-OH (NovaBiochem, Cat #04-12-1030, Lot A21653), Fmoc-Leu-OH (NovaBiochem, Cat #04-12-1025, Lot A25917), Fmoc-Met-OH (MidWest Biotech Cat #12400, Lot OP12240). Fmoc groups are removed prior to each coupling step with (2×10 min) treatments of 20% piperidine in DMF. All couplings are performed for 6 hours using an equal ratio of Fmoc amino acid, Diisopropylcarbodiimide (Sigma-Aldrich, Ca t #DI25407, Lot 80896APV) and HOAt (AK Scientific, Cat #D046, Lot 1188G50I), at a 3-fold molar excess over the theoretical peptide resin substitution at a final concentration of ˜0.2 M in DMF. After coupling the last amino acid and the removal of the N-terminal Fmoc group, the peptidyl resin is formylated by treatment with a 6 fold excess of 2,4,6-trichlorophenyl formate (TCI, Cat #T3121, Lot P8AFA-PE) dissolved in DMF with 200 μL of diisoprolylethylamine and reacted for 3 hrs at RT. The resin is then washed with DCM and diethyl ether and thoroughly dried by applying vacuum suction to the reaction vessel for 5 min. The dry resin is treated with 25 mL of cleavage cocktail (TFA:anisole:water:triisopropylsilane, 88:5:5:1 v/v) for 2 hrs at RT. The resin is filtered off, washed with twice with 5 mL of neat TFA, and the combined filtrates treated with 50 mL of cold diethyl ether to precipitate the crude peptide. The peptide/ether suspension is then centrifuged at 4000 rpm for 4 minutes to form a solid pellet, the ether is decanted, and the solid pellet triturated with ether 2 additional times and dried in vacuo for 30 min. The crude peptide is solubilized in 20% acetonitrile/water and purified by RP-HPLC on a C18 preparative column (Phenomenex, Luna Phenyl-Hexyl, 21×250 mm) with a linear gradient of acetonitrile in water with 0.1% HCl to yield the lyophilized peptide as an HCl salt (125 mg, 73% yield based on starting resin substitution). Purity was assessed using analytical RP-HPLC and found to be >99%. The molecular weight was determined by analytical electrospray MS. Calc: 565.7 Da, Obs: 565.3 Da (average molecular weight). The following ion was observed: 566.3 (M+1H).

The ε-amino group of the lysine is acylated as follows: the lyophilized peptide ˜50 mg (˜0.088 mmol) is dissolved in 5 mL of anhydrous DMF with the aid of a sonicator. In a separate scintillation vial, 74 mg (1.1 equivalents) of Mal-dPEG12-OH (QuantaBiodesign Cat #10285, Lot IH1-A1240-80) is activated with 29 mg (1.1 equivalents) of TSTU (OakWood Chemicals, Cat #024891, Lot 024891) and 61 μL (4 equivalents) of DIPEA in 1 mL of dry DMF for 25 min at RT. The activated Mal-PEG12-OH is added drop-wise to the solubilized peptide in DMF (1 mL) and 62 μL (5 equivalents) of triethylamine is added and the reaction was mixed at RT. After 1 hr, the reaction is stopped by the addition of cold diethyl ether. The solution is then split and transferred into two 50 ml conical tubes and more cold ether is added to further precipitate the peptide. The peptide/ether suspensions are then centrifuged at 4000 rpm for 4 minutes to form solid pellets, the ether is decanted, and the solid pellets are triturated with ether 2 additional times and dried in vacuo for 30 min. The combined crude peptide pellets are solubilized in 20% acetonitrile/water and purified by RP-HPLC on a C18 preparative column (Phenomenex, Luna Phenyl Hexyl 21×250 mm) with linear gradients of acetonitrile in water with 0.1% TFA to yield the lyophilized peptide as a TFA salt (44.4 mg, 38% yield based on starting material). Purity was assessed using analytical RP-HPLC and found to be >96%. The molecular weight was determined by analytical electrospray MS. Calc: 1316.6 Da, Obs: 1316.2 Da (average molecular weight). The following ions were observed: 659.0 (M+2H), and 1317.2 (M+1H). This peptide (formyl-Met-Leu-Phe-Lys (Mal-PEG12)-OH) can then be conjugated to an antibody as described in Example 3 below.

For unconjugated peptides used in the Examples below, the maleimido group is further hydrolyzed by incubating 20 mg of the product from step 1 in 2 mL of 40 mM Tris HCl buffer, pH 8.0, overnight at RT. After 18 hours, the solution is diluted with 10 ml of 20% acetonitrile/water and purified by RP-HPLC on a C18 preparative column (Phenomenex, Luna Phenyl Hexyl 21×250 mm) with a linear gradient of acetonitrile in water with 0.1% TFA to yield the lyophilized peptide as a TFA salt (6.4 mg, 32% yield based on starting material). Purity is assessed using analytical RP-HPLC and found to be >94%. The molecular weight is determined by analytical electrospray MS: Calc: 1334.6 Da; Obs: 1334.4 Da (average molecular weight). The following ions are observed: 668.0 (M+2H), and 1335.8 (M+1H).

### Example 2 (B): Synthesis of H-Met-Leu-Phe-Lys (Mal-PEG12-OH (“Peptide-'844”) (SEQ ID NO: 24)

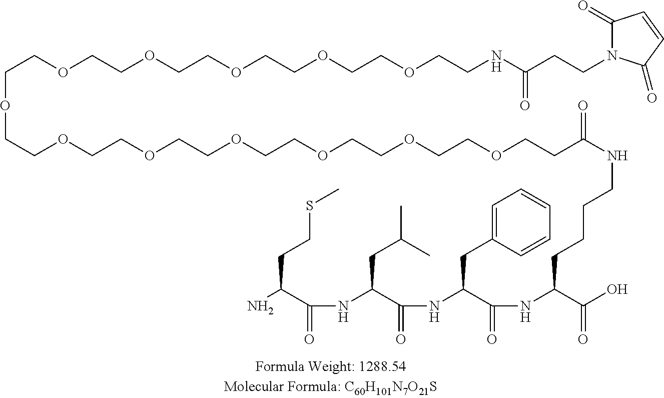 [SMILES: CSCC[C@H](N)C(=O)N[C@@H](CC(C)C)C(=O)N[C@@H](Cc1ccccc1)C(=O)N[C@@H](CCCCNC(=O)CCOCCOCCOCCOCCOCCOCCOCCOCCOCCOCCOCCOCCNC(=O)CCN1C(=O)C=CC1=O)C(=O)O]

Peptide-'844 with hydrolyzed maleimido group used as unconjugated peptide.

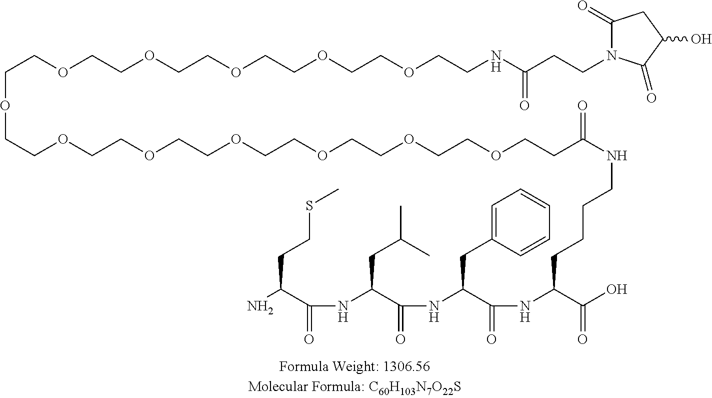 [SMILES: CSCC[C@H](N)C(=O)N[C@@H](CC(C)C)C(=O)N[C@@H](Cc1ccccc1)C(=O)N[C@@H](CCCCNC(=O)CCOCCOCCOCCOCCOCCOCCOCCOCCOCCOCCOCCOCCNC(=O)CCN1C(=O)CC(O)C1=O)C(=O)O]

A negative control peptide lacking formylation ((H-Met-LeuPhe-Lys-OH) (SEQ ID NO: 25) is produced by manual solid phase peptide synthesis using standard fluorenylmethoxycarbonyl (Fmoc)/tertiary butyl group (tBu) chemistry at a 0.3 mmol scale. Peptide assembly is done in a 100 mL fritted glass manual reaction vessel from Ace Glassware Inc. The solid support used for the synthesis is Fmoc-Lys (Mtt)-Wang resin, (NovaBiochem, Cat #8.56021, Lot S6692621 503), 100-200 mesh, with a substitution of 0.57 meq/g. Standard amino acids used are Fmoc-Phe-OH (NovaBiochem, Cat #04-12-1030, Lot A21653), Fmoc-Leu-OH (NovaBiochem, Cat #04-12-1025, Lot A25917), Fmoc-Met-OH (MidWest Biotech Cat #12400, Lot OP12240).

Fmoc groups are removed prior to each coupling step with (2×10 min) treatments of 20% piperidine in DMF. All couplings are performed for 6 hours using an equal ratio of Fmoc amino acid, diisopropylcarbodiimide (Sigma-Aldrich, Cat #DI25407, Lot 80896APV) and HOAt (AK Scientific, Cat #D046, Lot 1188G501), at a 3-fold molar excess over the theoretical peptide resin substitution and at a final concentration of ˜0.2 M in DMF.

After coupling the last amino acid and the removal of the N-terminal Fmoc group, the peptidyl resin is protected with a Boc (butyloxycarbonyl)-group by treatment with a 6 fold excess of Boc₂O (NovaBiochem, Cat #01-63-0007, Lot A25675) dissolved in dimethylformamide (DMF) with 200 μL of diisoprolylethylamine and reacted for 3 hrs at RT. The resin is then washed 8 times with dichloromethane (DCM) and the Mtt (4-methyltrityl) protecting group on the Lys residue was selectively removed with three consecutive treatments of 20% hexafluoroisopropanol (Oakwood Chemicals, Cat #003409) in DCM (2×10 min and 1×45 min) to expose the free epsilon amine of Lys for further reactions. Subsequent couplings of Fmoc PEG12-OH (BroadPharm, Cat #BP-22241) and 3-maleimido-propionic acid (Bachem, Cat #Q-2620) are done in the same fashion as the standard amino acid residues.

After the synthesis was complete, the peptidyl resin is washed with DCM, diethyl ether and thoroughly dried by applying vacuum suction to the reaction vessel for 5 min. The dry resin is treated with 25 mL of cleavage cocktail (trifluoroacetic acid (TFA):anisole:water:triisopropylsilane, 88:5:5:1 v/v) for 2 hrs at RT. The resin is filtered off, washed with twice with 5 mL of neat TFA, and the combined filtrates are treated with 50 mL of cold diethyl ether to precipitate the crude peptide. The peptide/ether suspension is then centrifuged at 4000 rpm for 4 minutes to form a solid pellet, the ether is decanted, and the solid pellet is triturated with ether 2 additional times and dried in vacuo for 30 min.

The crude peptide is solubilized in 20% acetonitrile/water and purified by RP-HPLC on a C18 preparative column (Phenomenex, Luna Phenyl-Hexyl, 21×250 mm) with a linear gradient of acetonitrile in water with 0.1% TFA to yield the lyophilized peptide as a TFA salt (38.8 mg, 10% yield based on starting resin substitution). Purity is assessed using analytical RP-HPLC and found to be >96%. The molecular weight is determined by analytical electrospray MS. Calc: 1288.5 Da, Obs: 1288.4 Da (average molecular weight). The following ions are observed: 645.0 (M+2H), and 1289.7 (M+1H). This peptide (H-Met-Leu-Phe-Lys (Mal-PEG12-OH) can then be conjugated to an antibody as described in Example 3 below.

For unconjugated peptides used in the Examples below, the maleimido group is further hydrolyzed by incubating 20 mg of the product from step 1 in 2 mL of 40 mM Tris HCl buffer, pH 8.0, overnight at RT. After 18 hours, the solution was diluted with 10 mL of 20% acetonitrile/water and purified by RP-HPLC on a C18 preparative column (Phenomenex, Luna Phenyl Hexyl 21×250 mm) with a linear gradient of acetonitrile in water with 0.1% TFA to yield the lyophilized peptide as a TFA salt (5.2 mg, 26% yield based on starting material). Purity was assessed using analytical RP-HPLC and found to be >96%. The molecular weight was determined by analytical electrospray MS. Calc: 1306.6 Da, Obs: 1306.4 Da (average molecular weight). The following ions were observed: 654.0 (M+2H), and 1307.7 (M+1H).

### Example 2 (c): Synthesis of formyl-Nle-Leu-Phe-PEG12-Lys (Maleimido-Propionyl)-OH (“fNle”; SEQ ID NO: 42)

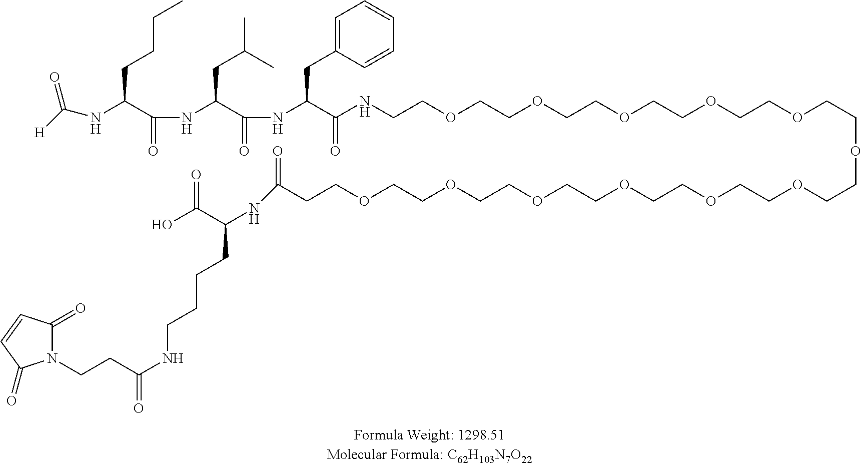 [SMILES: CCCC[C@H](NC=O)C(=O)N[C@@H](CC(C)C)C(=O)N[C@@H](Cc1ccccc1)C(=O)NCCOCCOCCOCCOCCOCCOCCOCCOCCOCCOCCOCCOCCC(=O)N[C@@H](CCCCNC(=O)CCN1C(=O)C=CC1=O)C(=O)O]

The chemotactic peptide formyl-Nle-Leu-Phe-PEG12-Lys-OH is synthsized as an an HCl salt (Peptides International) and is used for as a substrate for derivation without further modifications.

The acylation of the s-amino group of lysine is performed as follows: the lyophilized peptide ˜50 mg (˜0.044 mmol) is dissolved in 5 mL of anhydrous DMF with the aid of a sonicator. In a separate scintillation vial, 8.1 mg (1.1 equivalents) of maleimido-propionic acid (Bachem, Cat #Q-2620, Lot 0564230) is activated with 14.5 mg (1.1 equivalents) of TSTU (OakWood Chemicals, Cat #024891, Lot 024891) and 33.4 μL (4 equivalents) of DIPEA in 1 mL of dry DMF for 25 min at RT. The activated maleimido-propionic acid is added drop-wise to the solubilized peptide in DMF (1 mL) and then, 30 μL (5 equivalents) of triethylamine is added and the reaction mixed at RT. After 1 hr, the reaction is stopped by the addition of cold diethyl ether. The solution is then split and transferred into two 50 ml conical tubes and more cold ether is added to further precipitate the peptide. The peptide/ether suspensions are then centrifuged at 4000 rpm for 4 minutes to form solid pellets, the ether is decanted, and the solid pellets triturated with ether 2 additional times and dried in vacuo for 30 min. The combined crude peptide pellets are solubilized in 20% acetonitrile/water and purified by RP-HPLC on a C18 preparative column (Phenomenex, Luna Phenyl Hexyl 21×250 mm) with linear gradients of acetonitrile in water with 0.1% TFA to yield the lyophilized peptide as a TFA salt (8.6 mg, 15.1% yield based on starting material). Purity was assessed using analytical RP-HPLC and found to be >97%. The molecular weight was determined by analytical electrospray MS. Cal: 1298.5 Da, Obs: 1298.8 Da (average molecular weight). The following ions were observed: 650.0 (M+2H), and 1299.8 (M+1H). This peptide can then be conjugated to an antibody as described in Example 3 below.

### EXAMPLE 3

### Conjugation of IgG Antibodies to Peptides

Antibody-peptide bioconjugates may be prepared as follows. Parental antibody containing the engineered cysteine residues is buffer-exchanged into 50 mM tris(hydroxymethyl)aminomethane (Tris-HCl), 2 mM Ethylenediaminetetraacetic acid (EDTA), pH 7.5 using Zeba™ Spin Desalting Columns (40K MWCO) and brought to a final concentration of 5 mg/ml. A freshly prepared 100 mM Dithiothreitol (DTT) solubilized in MilliQ water is added in 40-fold molar excess to the antibody. The reaction mixture is incubated at room temperature for 16 hours. Following the incubation period, the reaction mixture is buffer exchanged into 50 mM tris (hydroxymethyl) aminomethane (Tris-HCl), 150 mM Sodium chloride (NaCl), pH 7.5 using Zeba Spin Desalting columns to remove excess unreacted DTT.

Freshly prepared 100 mM Dehydroascorbic acid (dHAA) in Dimethylacetamide is added in 30-fold molar excess to the antibody and incubated at room temperature for 3 hours. Following the incubation, 4-, 8-, or 12-fold molar excess of formyl-Met-Leu-Phe-Lys (Mal-PEG12)-OH (SEQ ID NO:22), H-Met-Leu-Phe-Lys (Mal-PEG12)-OH (SEQ ID NO: 24) or formyl-Nle-Leu-Phe-PEG12-Lys (Maleimido-Propionyl)-OH (synthesized as described in Examples 2(A), 2(B) and 2(C), respectively) is added (dissolved in Molecular grade water) to antibodies with one, two, or three engineered cysteine residues, respectively, to result in bioconjugates of 2, 4, or 6 ratios. This reaction mixture is incubated for 1 hour at room temperature. Post incubation, the sample is buffer exchanged into desired buffer and excess of unconjugated peptide is removed using desalting column, preparative size exclusion chromatography (pSEC), or dialysis.

Table 1 provides conjugated and unconjugated IgG antibody constructs prepared essentially as described herein and above, and tested in the assays that follow, including the antibody HC and LC sequences and the pegylated peptide used for conjugation. As used herein, “emibetuzumab”, “TMab” (trastuzumab), and “AME133” refer to antibody constructs containing the variable regions of the indicated antibody.

TABLE 1
Conjugated and unconjudated IgG antibody constructs.

| | Engineered | | |
|---|---|---|---|
| | cysteine | HC | LC |
| Construct | sites in each | SEQ ID | SEQ ID |
| Nomenclature ^{a, b, c} | HC or LC | NO: | NO: |
| Emibetuzumab-G4 | 378C | 1 | 5 |
| (PAA)-fMLFK- | | | |
| HC-378C | | | |
| Emibetuzumab-G4 | 124C | 2 | 5 |
| (PAA)-fMLFK- | | | |
| HC-124C | | | |
| Emibetuzumab-G4 | 124C | 3 | 5 |
| (PAA)-fMLFK- | 378C | | |
| HC-124C-378C | | | |
| Emibetuzumab-G4 | 124C | 4 | 5 |
| (PAA)-fMLFK- | 375C | | |
| HC-124C-375C | | | |
| Emibetuzumab-G4 | 124C | 3 | 5 |
| (PAA)-UC- | 378C | | |
| HC-124C-378C | | | |
| Emibetuzumab-G4 | 124C | 4 | 5 |
| (PAA)-UC- | 375C | | |
| HC-124C-375C | | | |
| Emibetuzumab-G4 | 378C | 1 | 5 |
| (PAA)-fNle- | | | |
| HC-378C | | | |
| Emibetuzumab-G4 | 124C | 2 | 5 |
| (PAA)-fNle- | | | |
| HC-124C | | | |
| TMab-G1-fMLFK- | 124C | 6 | 7 |
| HC-124C-378C | 378C | | |
| TMab-G1-UC-HC- | 124C | 6 | 7 |
| 124C-378C | 378C | | |
| Emibetuzumab-G4- | 124C | 49 | 5 |
| fMLFK-HC-124C- | 157C | | |
| 157C-378C | 378C | | |
| Emibetuzumab- | 124C | 50 | 5 |
| G4(PAA)-fMLFK- | 162C | | |
| HC-124C-162C- | 378C | | |
| 378C | | | |
| AME133-G1(IQ)- | 124C | 8 | 9 |
| fMLFK-HC-124C- | 378C | | |
| 378C | | | |
| AME133-G1(IQ)- | 124C | 8 | 9 |
| UC-HC-124C-378C | 378C | | |
| Tmab-G1(IQE)-HC- | 124C | 51 | 7 |
| 124C-378C | 378C | | |
| Emibetuzumab- | 157C | 44 | 5 |
| G4(PAA)-fMLFK- | | | |
| HC-157C | | | |
| Emibetuzumab- | 162C | 45 | 5 |
| G4(PAA)-fMLFK- | | | |
| HC-162C | | | |
| Emibetuzumab- | 262C | 46 | 5 |
| G4(PAA)-fMLFK- | | | |
| HC-262C | | | |
| Emibetuzumab- | 397C | 48 | 5 |
| G4(PAA)-fMLFK- | | | |
| HC-397C | | | |
| Emibetuzumab- | 415C | 26 | 5 |
| G4(PAA)-fMLFK- | | | |
| HC-415C | | | |
| Emibetuzumab- | 156C | 43 | 27 |
| G4(PAA)-fMLFK- | | | |
| LC-156C | | | |
| Emibetuzumab- | 171C | 43 | 28 |
| G4(PAA)-fMLFK- | | | |
| LC-171C | | | |
| Emibetuzumab- | 191C | 43 | 29 |
| G4(PAA)-fMLFK- | | | |
| LC-191C | | | |
| Emibetuzumab- | 193C | 43 | 30 |
| G4(PAA)-fMLFK- | | | |
| LC-193C | | | |
| Emibetuzumab- | 202C | 43 | 31 |
| G4(PAA)-fMLFK- | | | |
| LC-202C | | | |
| Emibetuzumab- | 208C | 43 | 32 |
| G4(PAA)-fMLFK- | | | |
| LC-208C | | | |
| Tmab-G1(IQE)-HC- | 124C | 33 | 7 |
| 124C-157C | 157C | | |
| Bispecific Antibody I | 124C | 34 | 58 |
| HCA-124C-378C | 378C | | |
| Bispecific Antibody I | 124C | 35 | 59 |
| HCB-124C-378C | 378C | | |

^{a} The first term refers to the parental antibody, the second term refers to the immunoglobulin isotype, the third term refers to the N-formyl peptide conjugated to the antibody with a Mal-PEG12 linker (wherein “UC” means unconjugated, and thus the antibody was not conjugated to a peptide), the fourth term refers to the heavy chain engineered cysteines, denoted by the residues of the fifth and sixth terms (if applicable). For example, emibetuzumab-G4-fMLFK-HC-378C means the the parent antibody was emibetuzumab, it is an IgG4 antibody, the N-formyl peptide used was fMLFK, and a cysteine was engineered in the heavy chain at position 378 (according to EU numbering).
^{b} antibody constructs labeled “(PAA)” contain additional mutations in the IgG4 constant region: 228P, 234A, and 235A (according to EU numbering).
^{c} antibody constructs labeled “(IQ)” contain additional mutations in the IgG1 constant region: 2471 and 339Q (according to EU numbering).

### EXAMPLE 4

### Conjugation Ratio Determination

Conjugation ratios for Peptide-'183 on the cysteine-engineered heavy chain of TMab (“trastuzumab”), AME133, and emibetuzumab constructs are determined by intact mass spec. analysis using the weighted average of the conjugate addition. Intact mass measurements are collected using an Agilent 1290 HPLC coupled to an Agilent 6230 ESI-TOF mass spectrometer. The sample (2 ug) is analyzed with a PLRP-S reversed phase column (Agilent) using a flow rate of 0.3 ml/min with water/0.2% formic acid as mobile phase A and acetonitrile/0.2% formic acid as mobile phase B with gradient elution from 20 to 70% B in 4 minutes. The Agilent 6230 TOF is run in positive ion mode at 4000V, skimmer at 65V, fragmentor at 300V, gas temperature at 350C, dry gas at 12 psi and nebulizer gas at 40 psi. The MS scan is from 600 m/z to 5000 m/z with a 1 scan/second. Data are collected from 2 minutes to 15 minutes and the protein molecular weight is determined by summing the TIC peak spectra followed by deconvolution with Agilent Mass Hunter and Bioconfirm v7.0. The deconvolution for the non-reduced sample is from 50000 to 190000 Da. with a peak width of 1.0 Da. 20 iterations and a 1 Da. step.

TABLE 2a
Peptide-′183:cysteine coniudation ratios.

| Sample ^{a} | Conjugation Ratio |
|---|---|
| Tmab-G1-fMLFK-HC-124C-378C | 3.82 |
| AME133-G1(IQ)-fMLFK-HC-124C-378C | 3.90 |
| Emibetuzumab-G4 (PAA)-fMLFK-HC-124C- | 4.11 |
| 378C | |
| Emibetuzumab-G4(PAA)-fMLFK-HC-124C- | 3.82 |
| 375C | |
| Emibetuzumab-G4(PAA)-fMLFK-HC-378C | 1.92 |

^{a} antibody constructs are designated according to the same convention as described inTable 1 of Example 1, herein.

Samples for serum stability are prepared by adding 50 μl of 1 mg/ml antibody conjugate to mouse serum and incubating at 37° C. for 0.5 to 48 hours with shaking at 300 RPM. All in vivo samples or serum stability samples require extraction from the biological matrix prior to the determination of the conjugation ratio. The biological fluid undergoes centrifugation at 13,000 RPM for 10 minutes followed by application to a Human Fc Select affinity column using a step gradient. The conjugated antibody is captured in mobile phase A (PBS, pH 7.4) and eluted with 0.2% (V/V) formic acid. Sample fractions are collected manually and dried to 50-100 μl using vacuum centrifugation with low heat. The percent off target denotes addition of the bioconjugate to sites other than the intended cysteine. Following the procedures described above, the following data were obtained.

TABLE 2b
Site specific conjugation with peptide-frm-MLFK
(Mal-PEG12)-OH (Peptide-'183) on cMet single
engineered cysteine mutants

| | | Conjugated | Off | Ring | | | | |
|---|---|---|---|---|---|---|---|---|
| | | antibody | target | open | Serum Stability | | | |
| | EU # | (CR) | (%) | (%) | 0 hr | 6 hr | 18 hr | 48 hr |
| 1 | HC124 | 1.88 | 8 | 87.1 | 1.9 | 1.9 | 1.2 | ND |
| 2 | HC157 | 1.93 | 8 | 42.5 | 1.9 | 1.9 | 1.7 | 0.7 |
| 3 | HC162 | 1.74 | 10 | 17.1 | 1.7 | 1.1 | 1.4 | 0.8 |
| 4 | HC262 | 0.62 | ND | ND | ND | ND | ND | ND |
| 5 | HC378 | 1.95 | 3 | 20.3 | 2.0 | 1.8 | 1.9 | 1.8 |
| 6 | HC397 | 0.36 | ND | ND | ND | ND | ND | ND |
| 7 | HC415 | 1.60 | 12 | 12 | 1.6 | 1.2 | 0.9 | 0.9 |
| 8 | LC156 | 2.02 | 5 | 43.5 | 2.0 | 2.0 | 1.5 | ND |
| 9 | LC171 | 1.97 | 13 | 7.1 | 2.0 | 2.0 | 1.9 | 1.7 |
| 10 | LC191 | 1.99 | 50 | 33 | 2.0 | 1.7 | 1.7 | 1.3 |
| 11 | LC193 | 1.65 | 50 | 39 | 2.0 | 2.0 | 1.6 | 1.3 |
| 12 | LC202 | 0.43 | ND | ND | ND | ND | ND | ND |
| 13 | LC208 | 1.78 | 10 | 34 | 1.6 | ND | 1.3 | 0.5 |

TABLE 2c
Site specific conjugation with peptide frm-Met-Ile-Phe-Leu-NH-(CH2)2-NH-[(Mal-Dap(NH2)]
(SEQ ID NO: 41; FRM-032) on cMet single engineered cysteine mutants

| | FRM- | Off | Ring | | | | | |
|---|---|---|---|---|---|---|---|---|
| | 032 | target | open | Serum Stability | | | | |
| EU # | (CR) | (%) | (%) | 0.5 hr | 2 hr | 6 hr | 24 hr | 48 hr |
| HC124 | 1.94 | 2.3 | >99 | 1.94 | 1.95 | 1.93 | 1.95 | 1.90 |
| HC157 | 1.95 | 1.6 | >99 | 1.9 | 1.7 | 1.7 | 1.7 | 1.6 |
| HC162 | 1.70 | 1.6 | >99 | 1.6 | 1.9 | 1.7 | 1.7 | 1.8 |
| HC378 | 0.91 | ND | >99 | 0.91 | 0.93 | 1.00 | 1.10 | 1.25 |
| HC415 | 1.78 | 0.02 | >99 | 1.7 | 1.9 | 1.9 | 1.9 | 1.9 |
| LC156 | 2.00 | 1.5 | >99 | 2.0 | 2.0 | 2.0 | 2.0 | 2.0 |
| LC171 | 1.66 | 0.01 | >99 | 1.7 | 1.6 | 1.7 | 1.7 | 1.7 |
| LC191 | 1.99 | 20-50 | >99 | 2.0 | 2.0 | 2.0 | 2.0 | 2.0 |
| LC193 | 1.55 | 2.1 | >99 | 1.6 | 1.6 | 1.6 | 1.8 | 1.8 |
| LC208 | 0.24 | 33 | >99 | ND | ND | ND | ND | ND |

ND: Not determined

These data demonstrate that the conjugation of monoclonal antibodies at engineered cysteine sites 124, 157, 375 and/or 378 with formylated peptides constructs via maleimide chemistry results in the peptide: antibody conjugation ratio that is predicted by the number of cysteines that were added to the antibody, as demonstrated by the percent off target.

### EXAMPLE 5

### TMab Bioconjugate Binding Human HER2

Binding of TMab to human HER2 is determined by ELISA using 96 well cell culture plates coated with human HER2. The plate is exposed to binding antibodies for 80 minutes, washed to remove unbound antibodies and incubated with secondary antibody for 50 minutes. The plate is washed before developing for 25 minutes at 37° C. Binding is measured with 96-well plate reader at O.D.560. Following procedures essentially described above, the following data were obtained.

TABLE 3
Binding of TMab to human HER2 (O.D.560).

| | | Tmab-G1- | Tmab-G1-UC- |
|---|---|---|---|
| Concentration | | (fMLFK-HC- | HC-124C- |
| (μg/ml) | TMab | 124C-378C ^{a} | 378C ^{a} |
| 10.00 | 1.212 | 1.167 | 1.218 |
| 3.33 | 1.156 | 1.055 | 1.127 |
| 1.11 | 0.977 | 0.978 | 0.935 |
| 0.37 | 0.762 | 0.716 | 0.686 |
| 0.12 | 0.468 | 0.419 | 0.385 |
| 0.04 | 0.221 | 0.198 | 0.200 |
| 0.01 | 0.114 | 0.104 | 0.102 |
| 0.00 | 0.069 | 0.066 | 0.064 |

^{a} antibody constructs are designated according to the same convention as described in Table 1 of Example 1, herein.

These data demonstrate that the binding of TMab to human Her2 is not impacted by modifying the heavy chain to introduce cysteines at sites 124 and 378, and is not impacted by conjugation of Peptide-'183 to the cysteine residues at sites 124 and 378.

### EXAMPLE 6

### PMN Chemotaxis

Chemotaxis is measured by observing primary human polymorphonuclear neutrophil (PMN) migration across transwell membranes (Corning #3415) towards antibody conjugates in a modified Boyden chamber assay. Approximately 2-4×10⁵ cells from neutrophil-enriched preparations are seeded in upper transwell chambers on membranes with 3.0 um pores. The lower transwell chambers contain solutions of buffer alone and fMLF (N-formyl-Met-Leu-Phe peptide as positive control) and experimental antibody bioconjugates. Some experiments also included fMLFK(Mal[OH]-PEG12)-OH (hydrolyzed Peptide-'183) and H-Met-Leu-Phe-Lys(Mal[OH]-PEG12-OH (hydrolyzed Peptide-'844) as a positive controls. Following seeding in transwells, cells are placed at 37° C. in a humidified incubator. After one hour, any cells in the upper chamber are removed, and the percentage of cells which successfully migrated to the lower chamber are quantified using CellTiter-Glo™ (Promega #G7571) according to manufacturer specified protocol. Percent migration is defined as (number of cells migrating to lower chamber/number of cells initially seeded). Cell numbers are determined using standard curves. All data are transformed to percent relative to the maximal fMLF response for each individual experiment.

### N-Formyl Modification is Required for Stimulating PMN Chemotaxis

To determine the ability of N-formyl modified peptides to induce PMN migration, primary human PMNs are exposed to peptides with or without N-formyl modifications, and PMN migration response is measured. Following procedures essentially as described above, PMNs responded maximally to fMLF, Peptide-'183, and Peptide-'844 at concentrations of 10 nM, 1 nM and 1 μM respectively (Table 4). Peptide-'844 is similar to Peptide-'183 except Peptide-'844 lacks the N-formyl group, and is 1000 fold less potent at inducing PMN migration, as indicated by dose response differences between Peptide-'183 and Peptide-'844. Values are given as percent PMN migration relative to 10 nM fMLF.

TABLE 4a
PMN migration towards fMLF, Peptide-′183, and Peptide-′844.
Relative Migration

| | | | Peptide-′183 | Peptide-′844 |
|---|---|---|---|---|
| Concentration | | fMLF | (SEQ ID NO: 22) | (SEQ ID NO: 24) |
| 1 | pM | 8.7 | 13.9 | 1.4 |
| 10 | pM | 17.3 | 5.5 | 4.1 |
| 100 | pM | 16.0 | 60.5 | 0.4 |
| 1 | nM | 86.1 | 103.4 | 11.1 |
| 10 | nM | 100.0 | 78.8 | 2.9 |
| 100 | nM | 67.3 | 22.0 | 20.4 |
| 1 | uM | 13.0 | 5.0 | 114.2 |
| 10 | uM | 6.9 | 12.9 | 110.4 |

These data demonstrate that N-formyl modification of the peptide is important for inducing PMN chemotaxis.

### Formyl Peptide Variants Induce Neutrophil Chemotaxis

Primary human neutrophils are exposed to formyl peptides and PMN migration response is measured essentially as described above except raw migration values are retained instead of being transformed into cell counts. Following procedures essentially as described above, the following data are provided as percent relative to 100 nM fMLF.

TABLE 4b
PMN Chemotaxis Towards Formyl Peptides

| | | Peptide- | | | | FRM- |
|---|---|---|---|---|---|---|
| | | '183 | | | | 031 |
| Concen- | | (SEQ | FRM-021 | FRM-029 | FRM-030 | (SEQ |
| tration | | ID NO: | (SEQ ID | (SEQ ID | (SEQ ID | ID NO: |
| (nM) | fMLF | 22) | NO: 36) | NO: 37) | NO: 38) | 39) |
| 1000 | 45.9 | 33.6 | 22.8 | 88.8 | 40.6 | 49.2 |
| 300 | 82.9 | 50.4 | 35.6 | 80.4 | 79.2 | 64.1 |
| 100 | 100.0 | 84.2 | 37.0 | 80.7 | 75.5 | 43.1 |
| 30 | n.d. | 118.4 | 52.9 | 112.0 | 59.0 | 73.2 |
| 10 | 98.9 | 145.4 | 137.4 | 110.9 | 80.4 | 98.8 |
| 3 | 32.7 | 167.0 | 142.2 | 176.0 | 134.2 | 145.5 |
| 1 | 66.7 | 149.8 | 151.7 | 106.7 | 142.8 | 157.7 |

n.d. = not determined

These data demonstrate that modifications to the formyl peptide amino acid sequence and linker can induce neutrophil migration mediated by FPR1. The PEG linked peptides [Peptide-'183, FRM-021, FRM-029, FRM-030, and FRM-031] maximally induced neutrophil migration at exposure concentrations between 1 and 3 nM.

### Role of N-Formyl Peptide Amino Acid Sequence and Conjugation Sites in Driving PMN Chemotaxis

A human anti-MET IgG4 antibody (emibetuzumab) is modified to include a cysteine residue at either CH1-S124 or CH3-A378 of each HC. Modified antibodies are conjugated to either Peptide-'183 or f-Nle (formyl-Nle-Leu-Phe-PEG12-Lys (Maleimido-Propionyl)-OH) at a ˜2:1 peptide to antibody ratio. Primary human PMNs are exposed to these different antibody conjugates, and PMN migration response is measured.

Antibody-peptide bioconjugates are as follows: emibetuzumab-G4-fMLFK-HC-378C, emibetuzumab-G4-fNle-HC-378C, emibetuzumab-G4-fMLFK-HC-124C, and emibetuzumab-G4-fNle-HC-124C.

Following procedures essentially as described above, the fNle conjugated antibodies were less potent at stimulating PMN migration than Peptide-'183 conjugated antibodies. Antibodies conjugated to Peptide-'183 at sites A378 and S124 maximally induced PMN migration at 30 nM, inducing migration responses equal to 99.1 and 117.8 percent of fMLF, control respectively. In contrast, the fNle antibody conjugates maximally induced PMN migration at 100 nM, resulting in migration responses equal to 71.7 and 76.5 percent of fMLF control respectively. The values below in Table 5 are given as percent PMN migration relative to 100 nM fMLF.

TABLE 5
PMN migration towards antibody conjugates.
Relative Migration

| | | Emibetu- | Emibetu- | Emibetu- | Emibetu- |
|---|---|---|---|---|---|
| | | zumab- | zumab- | zumab- | zumab- |
| | | G4(PAA)- | G4(PAA)- | G4(PAA)- | G4(PAA)- |
| | | fMLFK-HC- | fNle- | fMLFK-HC- | fNle- |
| Conc. | fMLF | 378C ^{a} | HC-378C ^{a} | 124C ^{a} | HC-124C ^{a} |
| 1 nM | 34.18 | 20.53 | 9.75 | 17.59 | 18.21 |
| 3 nM | 80.17 | 54.89 | 12.36 | 47.72 | 10.34 |
| 10 nM | 91.54 | 91.43 | 15.23 | 93.78 | 10.63 |
| 30 nM | 96.50 | 99.12 | 36.24 | 117.81 | 28.00 |
| 100 nM | 100.00 | 84.95 | 71.67 | 105.04 | 76.49 |
| 300 nM | 77.26 | 61.81 | 59.16 | 74.91 | 58.46 |
| 1 uM | 41.80 | 41.46 | 42.86 | 45.88 | 42.72 |

^{a} antibody constructs are designated according to the same convention as described in Table 1 of Example 1, herein.

These data demonstrate that antibodies conjugated to Peptide-'183 are significantly more potent than fNle antibody conjugates at inducing PMN migration. Both A378 and S124 sites are suitable for N-formyl peptide conjugation.

### Higher Peptide-to-Antibody Conjugation Ratios Increase PMN Migration Response

Human anti-MET IgG4 antibody (emibetuzumab) with amino acid modifications at CH1-124C and 378C or at 378C only is conjugated to Peptide-'183. Primary human PMNs are exposed to these antibody conjugates, and PMN migration response is measured.

Following procedures essential as described above, emibetuzumab-G4-fMLFK-HC-124C-378C maximally induced migration at 12.5 nM and emibetuzumab-G4-fMLFK-HC-378C maximally induced migration at 25 nM, inducing migration responses equal to 119.3 and 124.3 percent of fMLF control respectively (Table 6). Unconjugated antibody did not induce PMN migration relative to the conjugated antibodies. Values are given as percent PMN migration relative to 3.12 nM fMLF.

TABLE 6
PMN migration towards antibody conjugates.

| | Relative Migration | | | |
|---|---|---|---|---|
| | | Emibetuzumab- | Emibetuzumab- | Emibetuzumab- |
| | | G4(PAA)- | G4(PAA)- | G4(PAA)-UC- |
| Con- | | fMLFK-HC- | fMLFK- | HC-124C- |
| centration | fMLF | 124C-378C ^{a} | HC-378C ^{a} | 378C ^{a} |
| 0.78 nM | 45.39 | 26.10 | 16.25 | 5.48 |
| 1.56 nM | 86.79 | 35.98 | 18.39 | 8.05 |
| 3.12 nM | 100.00 | 74.75 | 36.56 | 7.24 |
| 6.25 nM | 99.26 | 105.07 | 74.37 | 6.36 |
| 12.5 nM | 87.60 | 119.29 | 111.52 | 4.11 |
| 25 nM | 94.77 | 117.40 | 124.30 | 5.70 |
| 50 nM | 95.36 | 109.62 | 98.48 | 12.02 |
| 100 nM | 79.69 | 91.16 | 88.86 | 6.67 |

^{a} antibody constructs are designated according to the same convention as described in Table 1 of Example 1, herein.

These data demonstrate that increasing the peptide to antibody ratio proportionally influences the PMN migration concentration response relationship.

TMab (trastuzumab) and AME133 Antibody Conjugates

TMab-G1-fMLFK-HC-124C-378C, AME133-G1(IQ)-fMLFK-HC-124C-378C, and emibetuzumab-G4-UC-124C-378C are studied in a PMN chemotaxis assay essentially as described above. TMab-G1-fMLFK-HC-124C-378C and AME133-G1(IQ)-fMLFK-HC-124C-378C maximally induced PMN migration at 10 nM and 3 nM respectively. Emibetuzumab-G4-UC-124C-378C did not induce PMN migration relative to conjugated antibodies. Values are given below in Table 7, and are a percent PMN migration relative to 30 nM fMLF.

TABLE 7
PMN migration towards antibody conjugates.

| | Relative Migration | | | |
|---|---|---|---|---|
| | | TMab-G1- | AME133-G1(IQ)- | Emibetuzumab- |
| | | fMLFK- | fMLFK-HC- | G4 (PAA)- |
| | | HC-124C- | 124C- | UC-124C- |
| Concentration | fMLF | 378C ^{a} | 378C ^{a} | 378C^{a} |
| 1 nM | 90.06 | 94.67 | 104.52 | 11.30 |
| 3 nM | 89.55 | 119.94 | 129.91 | 12.40 |
| 10 nM | 93.83 | 124.36 | 118.20 | 12.27 |
| 30 nM | 100.00 | 114.00 | 114.70 | 14.66 |
| 100 nM | 87.43 | 94.56 | 85.10 | 14.89 |
| 300 nM | 66.25 | 73.66 | 50.29 | 17.95 |

^{a} antibody constructs are designated according to the same convention as described in Table 1 of Example 1, herein.

These data demonstrate that TMab and AME133 antibodies conjugated to N-formyl peptides effectively induce PMN migration. Therefore, the conjugated antibodies of the present invention are believed to be useful for harnessing the body's immune system to attack cancer cells.

### EXAMPLE 7

### PMN Reactive Oxygen Species (ROS) Production

Polymorphonuclear neutrophils (PMN) are capable of producing ROS upon stimulation, and contain ROS producing enzymes like myeloperoxidase. Stimulation of PMNs induces degranulation and releases pre-formed ROS and ROS producing enzymes into the extracellular environment as a primary mechanism for responding to pathogens. Stimulation of ROS production by PMNs is sufficient for damaging and killing a wide range of targets, from bacteria to eukaryotic cells. One of the most effective pathways to stimulate PMNs to produce ROS involves engagement of formyl peptide receptor 1 (FPR1) on PMNs by N-formyl peptides. Fc-receptor engagement by antibodies on PMNs is also an effective mechanism to induce ROS production.

Production of ROS by human primary PMNs is measured using luminol-amplified chemiluminescence. Following isolation, PMNs are suspended at 1×10⁶ cells/ml in HBSS containing calcium and magnesium (Gibco #14025-092) supplemented with 0.25% human serum albumin (Gemini Bio producst #800-124) and 50 uM Luminol (SigmaAldrich #123072-2.5G). 100 μl of cell suspension (1×10⁵ total cells) is then distributed into each well of a 96-well plate suitable for fluorescence measurement (Greiner #655098) and temperature equilibrated to 37° C. for 5 minutes. Following equilibration, 10× solution of antibody conjugate is applied to the wells, achieving a 1× final concentration.

Immediately after the addition of antibody conjugate, chemiluminescence signal is recorded in a luminometer maintained at 37° C. with 0.01 seconds dwell time per well, 20 seconds total time between sequential plate readings and 45 minutes total run time (PerkinElmer EnVision Multilabel Plate Reader). Area under the curve (AUC) scores are calculated using luminescence signal from the first 5 minutes of each run, indicative of the relative amplitude of the initial ROS burst for each exposure condition. Formyl-Met-Leu-Phe (fMLF) peptide is used as a positive control, and cyclosporin H is used as an FPR1 inhibitor. Values are displayed as percent of fMLF control at maximal exposure concentration ((AUC Exposure Condition/AUC fMLF)×100).

Primary human PMNs were exposed to peptides or bioconjugates, and ROS production was measured using luminol amplified chemiluminescence essentially as described above. Following procedures essentially as described above, N-formyl peptides conjugated to monoclonal antibodies with the indicated engineered cysteine(s) effectively engage formyl peptide receptors expressed by primary human polymorphonuclear neutrophils and stimulate the production of cytotoxic reactive oxygen species. Stimulation of ROS production by conjugated N-formyl peptides was predominantly FPR1 dependent, as inhibition of FPR1 signaling by the FRP1 antagonist cyclosporin H significantly reduced PMN ROS production in response to N-formyl peptide conjugated antibodies. Examples using specific antibody conjugates are shown below.

### Peptide N-Formyl Modifications

Primary human PMNs were exposed to peptides, and ROS production was measured using luminol amplified chemiluminescence essentially as described above. Data are shown below in Table 8, and data are reported as percentage relative to 10 uM fMLF using area under curve calculations for luminescence recorded during the 5 minutes following exposure to antibody conjugates.

TABLE 8a
Stimulation of ROS Production by PMNs requires
peptides with N-Formyl modifications.

| | | Peptide-′183 | Peptide-′844 |
|---|---|---|---|
| Concentration | fMLF | (SEQ ID: 22) | (SEQ ID: 24) |
| 10 μM | 100 | 107 | 23.5 |
| 1 μM | 85.7 | 94 | 8.7 |
| 100 nM | 50.7 | 76.6 | 8.2 |
| 10 nM | 13.1 | 31.3 | 7.8 |
| 1 nM | 10 | 8.8 | 8.4 |
| 100 pM | 7.9 | 7.5 | 7.5 |
| 10 pM | 7.7 | 7.4 | 7.5 |
| 1 pM | 6.6 | 6.9 | 7.4 |

These data demonstrate that PMN's exposed to Peptide-'183 produced more ROS than observed for fMLF at concentrations from 10 nM to 10 uM. Peptide-'844 stimulated ROS production was substantially less than that observed for fMLF, indicating that peptide N-formyl modifications are required for effective stimulation of ROS production by PMNs.

### Formyl Peptide Variants Induce Neutrophil ROS Production

Primary human neutrophils were exposed to formyl peptide variants with amino acid substitutions, including synthetic amino acids, and ROS production was measured using luminol amplified chemiluminescence essentially as described above. Data are shown below in Table 8b, and data are reported as percentage relative to 3000 nM fMLF using area under curve calculations for luminescence recorded during the 5 minutes following exposure to reagents. EC50 values were calculated using Best-Fit values in Graphpad PRISM.

TABLE 8b
PMN ROS Production

| | Peptides | | | | | |
|---|---|---|---|---|---|---|
| | | Peptide- | | | | |
| | | '183 | | | | |
| Concen- | | (SEQ | FRM-021 | FRM-029 | FRM-030 | FRM-031 |
| tration | | ID NO: | (SEQ ID | (SEQ ID | (SEQ ID | (SEQ ID |
| (nM) | fMLF | 22) | NO: 36) | NO: 37) | NO: 38) | NO: 39) |
| 10000 | 88.8 | 89.1 | 91.7 | 118.3 | 133.8 | 117.2 |
| 3000 | 100.0 | 99.5 | 102.9 | 109.7 | 122.0 | 107.2 |
| 1000 | 83.6 | 94.3 | 91.2 | 92.9 | 103.7 | 97.6 |
| 300 | 66.7 | 76.2 | 84.4 | 81.8 | 86.8 | 93.6 |
| 100 | 36.5 | 49.9 | 69.2 | 46.7 | 67.4 | 75.6 |
| 30 | 11.9 | 12.2 | 34.4 | 7.8 | 36.9 | 27.0 |
| 10 | 4.8 | 3.6 | 8.4 | 2.6 | 5.5 | 6.5 |
| EC50 | 153.8 | 102.9 | 51.2 | 153.8 | 133.0 | 63.9 |

These data demonstrate the potency of the exemplified formyl peptide variants for inducing ROS production. It is anticipated that incorporation of a non-coded amino acid may improve peptide stability, and that non-coded amino acid variants could be incorporated to enhance engagement between the formyl peptide and FPR1, resulting in increased potency.

Mouse Neutrophil FPR-1 is More Sensitive to fMIFL Peptides and Antibody Conjugates than fMLF Derivatives

Mouse neutrophils purified from marrow were exposed to formyl peptides or antibody conjugates and ROS production was measured using luminol amplified chemiluminescence essentially as described above. Data are shown below in Table 8c, and data are reported as percentage relative to 10000 nM fMLF using area under curve calculations for luminescence recorded during the 5 minutes following exposure to reagents.

TABLE 8c
Mouse PMN ROS Production

| | Peptides and Antibody Conjugates | | | | |
|---|---|---|---|---|---|
| | | | | Tmab-G1- | Tmab-G1- |
| Con- | | | Tmab-G1- | fMLFK- | fMIFLK- |
| centration | | | UC-HC- | HC- | HC- |
| (nM) | fMLF | FRM-021 | 124C-378C | 124C-378C | 124C-378C |
| 10000 | 100.0 | 186.9 | Nd | nd | nd |
| 3000 | 70.6 | 194.4 | Nd | nd | nd |
| 1000 | 30.2 | 180.2 | 13.9 | 11.2 | 110.6 |
| 300 | 18.3 | 150.3 | 18.4 | 15.4 | 84.0 |
| 100 | 16.8 | 112.6 | 16.4 | 13.8 | 39.6 |
| 30 | 16.2 | 17.3 | 16.3 | 15.7 | 19.1 |
| 10 | 17.3 | 14.5 | 14.7 | 14.6 | 15.5 |
| 3 | nd | nd | 15.8 | 14.3 | 16.7 |
| 1 | nd | nd | 15.8 | 14.0 | 12.8 |

Nd = no data.

These data demonstrate that mouse neutrophils are significantly more sensitive to fMIFL peptides and antibody conjugates than fMLF variants. In humans, fMLF is one of the most potent FPR1 agonists while it is significantly less potent in mouse experiments. This relationship between FPR1 on mouse and human neutrophils holds true regardless of whether or not the FPR1 agonist is a soluble peptide or is conjugated to an antibody.

### TMab Bioconjugates

Primary human PMNs were exposed to TMab bioconjugates and ROS production was measured using luminol amplified chemiluminescence essentially as described above. Data are shown below in Table 9, and data are reported as percentage relative to 1000 nM fMLF using area under curve calculations for luminescence recorded during the 5 minutes following exposure to reagents.

TABLE 9
PMN ROS production.

| | Antibody Conjugates | | |
|---|---|---|---|
| | | TMab-G1-fMLFK- | TMab-G1-UC- |
| Concentration | fMLF | HC-124C-378C ^{a} | HC-124C-378C ^{a} |
| 1000 nM | 100.0 | 70.1 | 12.8 |
| 300 nM | 81.4 | 63.3 | 10.6 |
| 100 nM | 68.4 | 53.3 | 10.5 |
| 30 nM | 25.8 | 32.8 | 10.6 |
| 10 nM | 22.1 | 23.3 | 10.8 |
| 3 nM | 15.0 | 17.7 | 10.5 |
| 1 nM | 13.5 | 14.4 | 10.4 |

^{a} antibody constructs are designated according to the same convention as described in Table 1 of Example 1, herein.

These data demonstrate that PMNs exposed to 1000 nM TMab-G1-fMLFK-HC-124C-378C produced ROS at levels equal to 70.1% of fMLF control and at a much higher level than TMab-G1-UC-HC-124C-378C.

### Emibetuzumab Conjugates

Primary human PMNs were exposed to emibetuzumab conjugates, and ROS production was measured using luminol amplified chemiluminescence essentially as described above. Data are shown below in Table 10, and data are reported as percentage relative to 1000 nM fMLF using area under curve calculations for luminescence recorded during the 5 minutes following exposure to antibody conjugates.

TABLE 10
PMN ROS production.

| | Antibody Conjugates ^{a} | | | |
|---|---|---|---|---|
| | | Emibetuzumab- | Emibetuzumab- | Emibetuzumab- |
| | | G4(PAA)- | G4(PAA)- | G4(PAA)- |
| Con- | | fMLFK- | fMLFK- | UC-HC- |
| centration | fMLF | HC-124C-378C | HC-378C | 124C-378C |
| 1000 nM | 100 | 62.2 | 48.9 | 32.2 |
| 500 nM | 77.9 | 53.6 | 38.3 | 23 |
| 250 nM | 62.1 | 29 | 23.9 | 24.9 |
| 125 nM | 50.6 | 24.7 | 23.5 | 24.6 |
| 62.5 nM | 35.2 | 27.7 | 23.4 | 24.5 |
| 31.3 nM | 26.1 | 27.9 | 24.5 | 25.3 |
| 15.6 nM | 23.2 | 28.3 | 29.6 | 24.9 |
| 7.8 nM | 23.9 | 27.6 | 27.3 | 25.6 |
| 3.9 nM | 25.5 | 26.5 | 28.2 | 25 |
| 2 nM | 24.5 | 27.4 | 27.7 | 25.5 |
| 1 nM | 23.8 | 27.4 | 25.8 | 25.1 |

^{a} antibody constructs are designated according to the same convention as described in Table 1 of Example 1, herein.

These data demonstrate that PMNs exposed to 1000 nM Emibetuzumab-G4-fMLFK-HC-124C-378C and Emibetuzumab-G4-fMLFK-HC-378C produced ROS at levels equal to 62.2% and 48.9% of 1000 nM fMLF control, respectively. Exposure to 1000 nM Emibetuzumab-G4-UC-HC-124C-378C generated lower ROS production equal to only 32.2% of control.

### AME133 (Anti-CD20) Conjugates

Primary human PMNs were exposed to AME133 antibody conjugates, and ROS production was measured using luminol amplified chemiluminescence essentially as described above. Data are shown below in Table 11, and data are reported as percentage relative to 1000 nM fMLF using area under curve calculations for luminescence recorded during the 5 minutes following exposure to antibody conjugates.

TABLE 11
PMN ROS production.

| | Antibody Conjugates ^{a} | | |
|---|---|---|---|
| | | AME133-G1(IQ)- | AME133-G1(IQ)- |
| Con- | | fMLFK-HC- | UC-HC-124C- |
| centration | fMLF | 124C-378C | 378C |
| 1000 nM | 100.0 | 77.9 | 13.9 |
| 300 nM | 81.4 | 67.1 | 11.4 |
| 100 nM | 68.4 | 61.0 | 10.3 |
| 30 nM | 25.8 | 35.2 | 10.5 |
| 10 nM | 22.1 | 27.1 | 10.6 |
| 3 nM | 15.0 | 20.2 | 10.6 |
| 1 nM | 13.5 | 16.0 | 10.3 |

^{a} antibody constructs are designated according to the same convention as described in Table 1 of Example 1, herein.

These data demonstrate that PMNs exposed to 1000 nM AME133-G1(IQ)-fMLFK-HC-124C-378C and AME133-UC produced ROS at levels equal to 77.9% and 13.9% of control respectively.

### Antibody Conjugates and Inhibition of FPR1 Signaling

To determine if conjugated antibodies elicit more ROS production than unconjugated antibodies, ROS production is measured essentially as described above. All peptides are tested at 300 nM final concentration. PMNs are pre-incubated with 1 uM Cyclosporin H for 30 minutes prior to addition of peptides.

Buffer is HBSS containing calcium and magnesium (Gibco #14025-092) supplemented with 0.25% human serum albumin (Gemini Bio producst #800-124) and 50 uM Luminol (SigmaAldric #123072-2.5G). Values are reported in Table 12a below, and are expressed as a percentage relative to fMLF area under curve calculations for luminescence recorded during the 5 minutes following exposure to antibody conjugates.

TABLE 12a
PMN ROS production.

| | Exposure | |
|---|---|---|
| Antibody Conjugate ^{a} | Buffer | Cyclosporin H |
| fMLF | 100 | 20.6 |
| TMab-G1-fMLFK-HC-124C-378C | 82.4 | 23 |
| TMab-G1-UC-HC-124C-378C | 18.1 | 18.2 |
| AME133-G1(IQ)-fMLFK-HC-124C-378C | 95.7 | 31.3 |
| AME133-G1(IQ)-UC-HC-124C-378C | 25.3 | 19.7 |
| Buffer | 14 | 12.3 |

^{a} antibody constructs are designated according to the same convention as described in Table 1 of Example 1, herein.

These data demonstrate that antibodies conjugated to fMLFK elicit substantially more ROS production from human PMNs compared to unconjugated antibodies. The data also demonstrate that pre-treating the PMNs with the FPR1 antagonist cyclosporin H leads to a substantial reduction in ROS levels in the antibody bioconjugates, but not in the unconjugated controls.

Antibody Mutations that Enhance FcγR3 Binding Increases FPR1-Mediated ROS Production in Response to N-Formyl Peptide Bioconjugates

Primary human neutrophils are exposed to Tmab N-formyl peptide conjugates with or without mutations in the Fc region that increase affinity for FcγR3 (2471, 339Q, +/−332E mutations). ROS production is measured using luminol amplified chemiluminescence essentially as described above. Data are shown below in Table 12b, and data are reported as percentage relative to 1000 nM fMLF using area under curve calculations for luminescence recorded during the 5 minutes following exposure to reagents. EC₅₀ values for FPR1 mediated ROS production are calculated using Best-Fit values in Graphpad PRISM.

TABLE 12b
PMN ROS Production

| | | Antibody Conjugates^{a} | | | |
|---|---|---|---|---|---|
| | | | Tmab-G1- | Tmab-G1- | Tmab-G1- |
| Con- | | | fMLFK- | fMLFK-HC- | fMLFK-HC- |
| centration | | | HC-124C- | 124C-378C- | 124C- |
| (nM) | fMLF | Tmab | 378C | IQ^{b} | 378C-IQE^{c} |
| 1000 | 100.0 | 11.3 | 57.6 | 56.7 | 52.9 |
| 300 | 79.4 | 12.1 | 47.3 | 59.9 | 61.2 |
| 100 | 45.3 | 18.7 | 31.4 | 41.8 | 53.2 |
| 30 | 27.1 | 13.9 | 21.8 | 32.3 | 46.8 |
| 10 | 17.6 | 13.3 | 15.1 | 18.4 | 33.7 |
| 3 | 11.2 | 14.6 | 15.9 | 18.9 | 24.9 |
| 1 | 11.6 | 13.6 | 15.1 | 16.5 | 16.2 |
| EC50 | 333.9 | ud | 164.1 | 55.2 | 11.0 |

^{a}Antibody constructs are designated according to the same convention as described in Table 1 of Example 1, herein. Ud = undetermined.
^{b}Antibody constructs labeled “(IQ)” contain additional mutations in the IgG1 constant region: 247I and 339Q (according to EU numbering).
^{c}Antibody constructs labeled “(IQE)” contain additional mutations in the IgG1 constant region: 247I, 332E, and 339Q (according to EU numbering).

These data demonstrate that N-formyl-Met bioconjugates can be engineered to further enhance ROS production by optimizing FcR engagement by neutrophils. Fc optimized Tmab bioconjugates with the IQ and IQE amino acid substitutions enhanced stimulated ROS production by neutrophils relative to wild type Tmab IgG1 conjugates, with Tmab-G1-fMLFK-HC-124C-378C-IQ and Tmab-G1-fMLFK-HC-124C-378C-IQE variants showing improvement in EC₅₀ by 2.98 and 14.9 fold when compared to Tmab-G1-fMLFK-HC-124C-378C respectively. It is anticipated that Fc-engineered improvements in activation of PMN cell killing mechanisms would convey substantial benefit in conjugated antibody-mediated cell killing by neutrophils.

Compound Linker Lengths Primary human neutrophils are exposed to N-formyl peptide Tmab conjugates with PEG linkers of varying lengths, and ROS production was measured using luminol amplified chemiluminescence essentially as described above. Data are shown below in Table 12c, and data are reported as percentage relative to 3000 nM FRM-023 (SEQ ID NO: 40) using area under curve calculations for luminescence recorded during the 5 minutes following exposure to reagents. EC50 values for FPR1-mediated ROS production were calculated using Best-Fit values in Graphpad PRISM.

TABLE 12c
PMN ROS Production

| | | Antibody Conjugates^{a} | | |
|---|---|---|---|---|
| | | Tmab-G1- | Tmab-G1- | Tmab-G1- |
| | | fMIFL- | fMIFL- | fMIFL- |
| | | HC124C- | HC124C- | HC124C- |
| Concentration | FRM- | 378C- | 378C- | 378C- |
| (nM) | 023 | (PEG12) | (PEG6) | (PEG3) |
| 10000 | 92.9 | ND | ND | ND |
| 3000 | 100.0 | ND | ND | ND |
| 1000 | 75.6 | 56.0 | 65.6 | 61.6 |
| 300 | 93.2 | 64.0 | 85.1 | 62.4 |
| 100 | 47.5 | 48.6 | 84.4 | 72.0 |
| 30 | 55.0 | 26.5 | 67.4 | ND |
| 10 | 18.0 | 11.5 | 26.7 | 38.9 |
| 3 | ND | 11.9 | 4.9 | 6.0 |
| 1 | ND | 12.0 | 7.5 | 5.4 |
| EC50 | 44.23 | 36.4 | 13.56 | 31.32 |

^{a}Antibody constructs are designated according to the same convention as described in Table 1 of Example 1, herein. ND = Not Determined.

These data demonstrate that N-formyl peptide conjugates maintain functionality as FPR1 agonists with varying sizes of PEG.

### EXAMPLE 8

### Antibody Conjugates Enable Neutrophil-Mediated Tumor Cell Killing

The ability of the antibody compounds to target PMNs to tumors and engage in tumor cell killing is determined. TMab, emibetuzumab, and AME133 antibody conjugates are assessed in solid tumors and in liquid tumors for their ability to engage PMNs in tumor cell killing.

Antibody-targeted killing of tumor cells by PMNs is measured using the xCelligence Real Time Cell Analysis system (ACEA Biosciences). This system monitors cell viability in real time by recording electrical impedance between sensors on the growth surface of culture plates. It reports a normalized cell index (NCI) that is normalized to control cells in parallel wells and allows one to control for relative culture viability. NCIs are measured continuously at 15 minute intervals for 24 hours following incubation of tumor cultures with targeted antibodies and addition of human primary PMNs at a 10:1 PMN to tumor cell ratio. Prior to seeding with tumor cells, xCelligence 96-well E-Plates are calibrated for background signal. Each well receives 50 μl of culture medium (RPMI+10% FBS+antibiotics) and the E-plate is equilibrated to 37° C. in a humidified incubator containing the xCelligence plate reader.

After equilibration, E-Plate well variations in background are measured. Cultured tumor cell lines are dissociated, counted and diluted to a final density of 1×10⁵ cells/ml in culture medium and 100 μl of diluted tumor cells were plated into E-Plate wells. The E-Plate is returned to the xCelligence reader and cell indices are measured in 15 minute intervals overnight to establish baseline.

The next day, PMNs are isolated from fresh human blood samples and brought to a final density of 2×10⁶ cells/ml in culture medium. Following overnight recording, the E-Plate is removed from the xCelligence reader and 22 μl of 10× antibody solution or buffer is added to designated wells. After 15 minutes, 50 μl of diluted PMNs (1×10⁵ total cells) or buffer was added to designated wells. Immediately after PMN addition, the E-Plate is returned to the xCelligence reader and cell indices were measured for up to 72 hours. After completion of the experiment, cell indices are normalized (NCI) to the time point immediately preceding the addition of antibodies.

Percent NCI is defined as ((NCI of sample)/(NCI of Tumor Cells Alone)×100). For non-adherent tumor cells (Daudi cells), the xCelligence Immunotherapy Kit-B Cell Killing Assay (ACEA #8100004) is used to tether the tumor cells to E-Plate wells according to manufacturer protocols. Following tethering and background acquisition, the protocols are performed as indicated above.

The data shown below demonstrate that antibodies conjugated to N-formyl peptides lead to PMN-mediated killing of tumor cells.

### N-formyl-Met-Leu-Phe Peptides

Two N-formylated peptides, f-Met-Leu-Phe and Peptide-'183 are evaluated in SKOV3 tumor cell killing assays to determine the impact of N-formyl methionine peptides on PMN mediated tumor cell killing in the absence of tumor targeting with monoclonal antibodies.

Percent NCI values represent relative viability of SKOV3 cells following 2 hours of exposure to the stated conditions. Values are given as mean percentage normalized to SKOV3 control±SD; n=4 for all conditions. Statistical significance is determined by one-way ANOVA followed by post-hoc Dunnett's multiple comparisons test vs “+ PMN”.

TABLE 13
Soluble formyl-peptides enhance PMN-mediated
killing of SKOV3 tumor cells.

| Exposure Condition | Percent NCI | P Value |
|---|---|---|
| +PMN | 104.5 ± 1.7 | |
| Buffer Control | 100 ± 1.5 | 0.3755 |
| f-MLF (3 nM) + PMN | 104 ± 1.3 | 0.9997 |
| f-MLF (10 nM) + PMN | 102.4 ± 1.2 | 0.9987 |
| f-MLF (30 nM) + PMN | 99.3 ± 1.3 | 0.4810 |
| f-MLF (100 nM) + PMN | 97.5 ± 3 | 0.1125 |
| f-MLF (300 nM) + PMN | 95.5 ± 1.2 | 0.0117 |
| f-MLF (3 nM) | 101.2 ± 2.5 | 0.9703 |
| f-MLF (10 nM) | 100.3 ± 0.9 | 0.7958 |
| f-MLF (30 nM) | 100.9 ± 0.7 | 0.9413 |
| f-MLF (100 nM) | 100.2 ± 1.5 | 0.7790 |
| f-MLF (300 nM) | 99.8 ± 0.8 | 0.6552 |
| Peptide-′183 (3 nM) + PMN | 100.4 ± 1 | 0.8229 |
| Peptide-′183 (10 nM) + PMN | 98.2 ± 0.7 | 0.2070 |
| Peptide-′183 (30 nM) + PMN | 97.5 ± 1.6 | 0.1118 |
| Peptide-′183 (100 nM) + PMN | 96.4 ± 0.8 | 0.0362 |
| Peptide-′183 (300 nM) + PMN | 92.5 ± 1.4 | 0.0001 |
| Peptide-′183 (3 nM) | 98.9 ± 2.2 | 0.3643 |
| Peptide-′183 (10 nM) | 99.5 ± 0.5 | 0.5344 |
| Peptide-′183 (30 nM) | 97.9 ± 2.3 | 0.1656 |
| Peptide-′183 (100 nM) | 100 ± 0.8 | 0.7180 |
| Peptide-′183 (300 nM) | 99.5 ± 1.1 | 0.5295 |

These data demonstrate that the peptides had no statistical impact on tumor cell viability in the absence of PMN. In the presence of PMN, these peptides caused reductions in NCI only at the highest concentrations of peptide.

### TMab

Adherent HER2(+) SKOV3 human adenocarcinoma tumor cells were plated for approximately 24 hrs, and then incubated with TMab-G1-fMLFK-HC-124C-378C or TMab-G1-UC-HC-124C-378C, and exposed to primary human PMNs at a 10:1 effector target to cell ratio.

The percent NCI values represent relative viability of SKOV3 cells following 2 hours of exposure to the stated conditions. Values are given below in Table 14, and are expressed as mean percentage normalized to SKOV3 control±SD. N=4 for all conditions.

TABLE 14
TMab conjugate PMN-mediated killing of SKOV3 tumor cells.

| Exposure Condition ^{a} | Percent NCI | P Value |
|---|---|---|
| +PMN | 104.5 ± 2.1 | |
| Buffer Control | 100 ± 1.7 | 0.3755 |
| TMab-G1-UC-HC-1240-378C | 103.3 ± 0.8 | 0.9994 |
| (3 nM) + PMN | | |
| TMab-G1-UC-HC-124C-378C | 103 ± 1.2 | 0.9991 |
| (10 nM) + PMN | | |
| TMab-G1-UC-HC-124C-378C | 103 ± 0.7 | 0.9992 |
| (30 nM) + PMN | | |
| TMab-G1-UC-HC-124C-378C | 103.1 ± 1 | 0.9993 |
| (100 nM) + PMN | | |
| TMab-G1-UC-HC-124C-378C | 102.8 ± 1.4 | 0.9991 |
| (300 nM) + PMN | | |
| TMab-G1-UC-HC-124C-378C (3 nM) | 100.4 ± 0.8 | 0.821 |
| TMab-G1-UC-HC-124C-378C (10 nM) | 100.4 ± 1 | 0.8337 |
| TMab-G1-UC-HC-124C-378C (30 nM) | 101.5 ± 0.4 | 0.9846 |
| TMab-G1-UC-HC-124C-378C (100 nM) | 99.4 ± 0.5 | 0.5015 |
| TMab-G1-UC-HC-124C-378C (300 nM) | 99.5 ± 0.7 | 0.5273 |
| TMab-G1-fMLFK-HC-124C-378C | 71.6 ± 8.3 | 0.0001 |
| (3 nM) + PMN | | |
| TMab-G1-fMLFK-HC-124C-378C | 63.5 ± 9.9 | 0.0001 |
| (10 nM) + PMN | | |
| TMab-G1-fMLFK-HC-124C-378C | 69 ± 8.2 | 0.0001 |
| (30 nM) + PMN | | |
| TMab-G1-fMLFK-HC-124C-378C | 76.3 ± 16.7 | 0.0001 |
| (100 nM) + PMN | | |
| TMab-G1-fMLFK-HC-124C-378C | 81.6 ± 12.1 | 0.0001 |
| (300 nM) + PMN | | |
| TMab-G1-fMLFK-HC-124C-378C (3 nM) | 101.8 ± 0.3 | 0.9982 |
| TMab-G1-fMLFK-HC-124C-378C (10 nM) | 101.5 ± 0.9 | 0.9857 |
| TMab-G1-fMLFK-HC-124C-378C (30 nM) | 101.6 ± 0.6 | 0.9859 |
| TMab-G1-fMLFK-HC-124C-378C (100 nM) | 101.1 ± 0.5 | 0.9638 |
| TMab-G1-fMLFK-HC-124C-378C (300 nM) | 100.9 ± 0.3 | 0.9334 |

^{a} antibody constructs are designated according to the same convention as described in Table 1 of Example 1, herein.

These data demonstrate that after 2 hrs, cells incubated with 10 nM TMab-G1-fMLFK-HC-124C-378C and exposed to PMNs showed diminished normalized cell index (NCI) equal to 63.5±9.9% percent of control cells (p-value<0.0001) while cells exposed to 10 nM TMab-G1-UC-HC-124C-378C maintained an NCI of 103±1.2% of control cells (not statistically significant). TMab-G1-fMLFK-HC-124C-378C did not reduce tumor cell viability after two hours in the absence of PMNs, and the addition of PMNs without antibody did not affect SKOV3 tumor cell viability.

### Emibetuzumab

Adherent MET(+) A549 human lung carcinoma cells are plated for approximately 24 hours, then incubated with Emibetuzumab-G4-fMLFK-HC-124C-375C or emibetuzumab-G4-UC-HC-124C-375C and exposed to primary human PMNs at 10:1 effector to target cell ratio.

Following procedures essentially as described above, the following data were obtained and are shown in Table 15.

TABLE 15
Formyl-peptide conjugated emibetuzumab-G4-fMLFK-HC-124C-375C
antibody enhances PMN mediated killing of A549 tumor cells.

| Exposure Condition ^{a} | Percent NCI | P Value |
|---|---|---|
| +PMN | 101 ± 1.9 | |
| A549 Control | 100 ± 0.8 | 0.9946 |
| Emibetuzumab-G4(PAA)-UC-HC- | 102.3 ± 1.7 | 0.9651 |
| 124C-375C (3 nM) + PMN | | |
| Emibetuzumab-G4(PAA)-UC- | 102.5 ± 1.9 | 0.899 |
| HC-124C-375C (10 nM) + PMN | | |
| Emibetuzumab-G4(PAA)-UC- | 102.7 ± 1.5 | 0.7836 |
| HC-124C-375C (30 nM) + PMN | | |
| Emibetuzumab-G4(PAA)-UC- | 102.9 ± 1.5 | 0.6665 |
| HC-124C-375C (100 nM) + PMN | | |
| Emibetuzumab-G4(PAA)-UC- | 102.3 ± 1.8 | 0.9651 |
| HC-124C-375C (300 nM) + PMN | | |
| Emibetuzumab-G4(PAA)-UC-HC- | 101.3 ± 0.7 | 0.9996 |
| 124C-375C (3 nM) | | |
| Emibetuzumab-G4(PAA)-UC- | 100.8 ± 1 | 0.9998 |
| HC-124C-375C (10 nM) | | |
| Emibetuzumab-G4(PAA)-UC- | 100.1 ± 0.9 | 0.9989 |
| HC-124C-375C (30 nM) | | |
| Emibetuzumab-G4(PAA)-UC- | 100.8 ± 1.4 | 0.9998 |
| HC-124C-375C (100 nM) | | |
| Emibetuzumab-G4(PAA)-UC- | 102.8 ± 4.9 | 0.7695 |
| HC-124C-375C (300 nM) | | |
| Emibetuzumab-G4(PAA)-fMLFK- | 94.8 ± 2.1 | 0.0001 |
| HC-124C-375C (3 nM) + PMN | | |
| Emibetuzumab-G4(PAA)-fMLFK- | 87.7 ± 0.9 | 0.0001 |
| HC-124C-375C (10 nM) + PMN | | |
| Emibetuzumab-G4(PAA)-fMLFK- | 89.6 ± 1.4 | 0.0001 |
| HC-124C-375C (30 nM) + PMN | | |
| Emibetuzumab-G4(PAA)- | 94.1 ± 0.4 | 0.0001 |
| fMLFK-HC-124C-375C | | |
| (100 nM) + PMN | | |
| Emibetuzumab-G4(PAA)- | 92.4 ± 0.9 | 0.0001 |
| fMLFK-HC-124C-375C | | |
| (300 nM) + PMN | | |
| Emibetuzumab-G4(PAA)-fMLFK- | 102.2 ± 0.5 | 0.9831 |
| HC-124C-375C (3 nM) | | |
| Emibetuzumab-G4(PAA)-fMLFK- | 101.8 ± 0.7 | 0.9988 |
| HC-124C-375C (10 nM) | | |
| Emibetuzumab-G4(PAA)-fMLFK- | 101.6 ± 0.8 | 0.9992 |
| HC-124C-375C (30 nM) | | |
| Emibetuzumab-G4(PAA)-fMLFK- | 101.5 ± 0.4 | 0.9994 |
| HC-124C-375C | | |
| (100 nM) | | |
| Emibetuzumab-G4(PAA)- | 102.2 ± 1.4 | 0.9811 |
| fMLFK-HC-124C-375C | | |
| (300 nM) | | |

^{a} antibody constructs are designated according to the same convention as described in Table 1 of Example 1, herein.

Percent NCI values represent relative viability of A549 cells following 2 hours of exposure to the stated conditions. Values are given as mean percentage normalized to “+PMN” control±SD; n=4 for all conditions. Statistical significance was determined by one-way ANOVA followed by post-hoc Dunnett's multiple comparisons test vs “+PMN”. NCI, normalized cell index; PMN, primary human polymorphonuclear neutrophils; ns, not significant.

These data demonstrate that cultures exposed to 10 nM emibetuzumab-G4-fMLFK-HC-124C-375C in the presence of PMNs showed reduced NCI equal to 87.7±0.9% of control cells after 2 hrs incubation, while emibetuzumab-G4-UC-HC-124C-375C treated cells maintained an NCI 102.5±1.9% of control cells.

### AME133 Example

Non-adherent, CD20+ Daudi B lymphoblast cells are immobilized with xCelligence Immunotherapy Kit (ACEA #8100004) to tether the tumor cells to E-Plate wells according to manufacturer protocols, and are exposed to conditions shown below in Table 16. Percent NCI values represent relative viability of DAUDI cells following 6 hours of exposure to the stated conditions. Values are given as mean percentage normalized to “Buffer control” ±SD; n=4 for all conditions. Statistical significance was determined by one-way ANOVA followed by post-hoc Dunnett's multiple comparisons test vs “+PMN”.

TABLE 16
Formyl-peptide conjugated AME133 antibody enhances
PMN mediated killing of DAUDI tumor cells.

| Exposure Condition ^{a} | Percent NCI | P Value |
|---|---|---|
| +PMN | 66.9 ± 5.2 | |
| Buffer Control | 100 ± 1.4 | 0.0001 |
| AME133-G1(IQ)-UC-124C-378C | 58.7 ± 13.2 | 0.6577 |
| (10 nM) + PMN | | |
| AME133-G1(IQ)-UC-124C-378C | 93.4 ± 22.4 | 0.0001 |
| (30 nM) + PMN | | |
| AME133-G1(IQ)-UC-124C-378C | 114 ± 6.9 | 0.0001 |
| (100 nM) + PMN | | |
| AME133-G1(IQ)-UC-124C-378C | 113.2 ± 7.2 | 0.0001 |
| (300 nM) + PMN | | |
| AME133-G1(IQ)-UC-124C-378C | 97.7 ± 1.4 | 0.0001 |
| (10 nM) | | |
| AME133-G1(IQ)-UC-124C-378C | 97.3 ± 0.6 | 0.0001 |
| (30 nM) | | |
| AME133-G1(IQ)-UC-124C-378C | 90.9 ± 0.6 | 0.0003 |
| (100 nM) | | |
| AME133-G1(IQ)-UC-124C-378C | 87.7 ± 1.5 | 0.0022 |
| (300 nM) | | |
| AME133-G1(IQ)-fMLFK-124C- | 27.4 ± 1 | 0.0001 |
| 378C (10 nM) + PMN | | |
| AME133-G1(IQ)-fMLFK-124C- | 20 ± 2.1 | 0.0001 |
| 378C (30 nM) + PMN | | |
| AME133-G1(IQ)-fMLFK-124C- | 42.6 ± 4.4 | 0.0003 |
| 378C (100 nM) + PMN | | |
| AME133-G1(IQ)-fMLFK-124C- | 84.5 ± 7.6 | 0.0141 |
| 378C (300 nM) + PMN | | |
| AME133-G1(IQ)-fMLFK-124C- | 102.5 ± 4.6 | 0.0001 |
| 378C (10 nM) | | |
| AME133-G1(IQ)-fMLFK-124C- | 103 ± 2.3 | 0.0001 |
| 378C (30 nM) | | |
| AME133-G1(IQ)-fMLFK-124C- | 93.3 ± 1.2 | 0.0001 |
| 378C (100 nM) | | |
| AME133-G1(IQ)-fMLFK-124C- | 89 ± 4 | 0.001 |
| 378C (300 nM) | | |

^{a} antibody constructs are designated according to the same convention as described in Table 1 of Example 1, herein.

These data demonstrate that cultures exposed to 30 nM AME133-G1(IQ)-fMLFK-124C-378C had reduced NCI equal to 20±2.1% of control cells (p-value <0.0001) after 6 hrs incubation, while cultures incubated with 30 nM AME133-G1(IQ)-UC-124C-378C maintained an NCI of 97.3±1.2% of control cells. AME133-G1(IQ)-fMLFK-124C-378C and AME133-G1(IQ)-UC-124C-378C did not reduce tumor cell viability in the absence of PMNs. However, exposure of Daudi cells to PMNs in the absence of antibody reduced tumor culture NCI to 66.9±5.2% of control cells (p-value <0.0001).

### Conjugation of Formyl Peptides to Multiple Cysteines of A Single Antibody Conjugate Increases Potency

Primary human neutrophils are exposed to IgG4 antibody conjugates with different numbers of engineered cysteine conjugation sites and ROS production is measured using luminol amplified chemiluminescence essentially as described above. Following procedures essentially as described above, the following data were obtained.

TABLE 17
PMN ROS Production.

| | Antibody Conjugates | | | | | |
|---|---|---|---|---|---|---|
| | | | G4- | G4- | | |
| | G4- | G4- | fMLFK- | fMLFK- | | |
| Concen- | fMLFK- | fMLFK- | 124C- | 124C- | G4- | |
| tration | HC- | 124C- | 162C- | 157C- | 124C- | |
| (nM) | 378C | 378C | 378C | 378C | 378C | fMLF |
| 1000 | 2.8 | 25.3 | 42.3 | 51.8 | 0.5 | 100.0 |
| 300 | 2.4 | 9.1 | 54.2 | 58.7 | 0.7 | 75.2 |
| 100 | 1.0 | 3.4 | 52.7 | 63.6 | 0.5 | 46.8 |
| 30 | 1.2 | 1.8 | 38.4 | 50.0 | 0.6 | 20.3 |
| 12 | 0.9 | 0.9 | 17.8 | 26.9 | 0.5 | 5.5 |
| 3 | 1.8 | 0.8 | 6.1 | 11.9 | 0.6 | 2.2 |
| 1 | 0.9 | 0.6 | 1.2 | 1.7 | 0.5 | 0.9 |

Data in Table 17 are reported as percentage relative to 1000 nM fMLF using area under curve calculations for luminescence recorded during the 5 minutes following exposure to reagents.

These data demonstrate that an antibody conjugated to fMLFK can be made more potent with additional sites of conjugation.

### ILLUSTRATIVE EMBODIMENTS

The following comprises a list of illustrative embodiments according to the instant disclosure which represent various embodiments of the instant disclosure. These illustrative embodiments are not intended to be exhaustive or limit the disclosure to the precise forms disclosed, but rather, these illustrative embodiments are provided to aide in further describing the instant disclosure so that others skilled in the art may utilize their teachings.
1. An antibody comprising an IgG heavy chain constant region and light chain constant region wherein said antibody comprises a cysteine at at least one of the following residues: residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain.
2. The antibody of embodiment 1, wherein said antibody comprises a cysteine at residue 124 in the C_{H}1 domain and further comprises a cysteine at one, but not all, of residue 157 and 162 in the C_{H}1 domain and residues 375 and 378 in the C_{H}3 domain.
3. The antibody of embodiment 1 or 2, wherein said antibody comprises a cysteine at residue 157 in the C_{H}1 domain.
4. The antibody of embodiment 2, wherein said antibody comprises a cysteine at residue 375 in the C_{H}3 domain.
5. The antibody of embodiment 2, wherein said antibody comprises a cysteine at residue 378 in the C_{H}3 domain.
6. An antibody of any one of embodiments 1 to 4 wherein said IgG heavy chain constant region is a human, mouse, rat, or rabbit IgG constant region.
7. The antibody of embodiment 5 wherein said IgG heavy chain constant region is a human IgG1 or human IgG4 isotype.
8. The antibody of embodiment 6 wherein said IgG heavy chain constant region is a human IgG1.
9. The antibody of embodiment 1 wherein the heavy chain constant region is human IgG1 given by the amino acid sequence of SEQ ID NO: 17, 18, 19, or 52.
10. The antibody of embodiment 2 wherein the heavy chain constant region is human IgG1 given by the amino acid sequence of SEQ ID NO: 20, 21, or 53.
11. An antibody according to any one of embodiments 7 to 9 wherein said IgG1 heavy chain constant region further comprises an isoleucine substituted at residue 247, a glutamine substituted at residue 339, and optionally a glutamic acid substituted at residue 332.
12. The antibody of embodiment 6 wherein said IgG heavy chain constant region is a human IgG4.
13. The antibody of embodiment 1 wherein the heavy chain constant region is human IgG4 given by the amino acid sequence of SEQ ID NO: 12, 13, 14, 54, or 55.
14. The antibody of embodiment 2 wherein the heavy chain constant region is human IgG4 given by the amino acid sequence of SEQ ID NO: 15, 16, 56, or 57.
15. An antibody according to anyone of embodiments 11 to 13 wherein said IgG4 heavy chain constant region further comprises a proline substituted at residue 228, an alanine substituted at residue 234, and an alanine substituted at residue 235 and a glutamine substituted at residue 339.
16. An antibody according to embodiment 1 comprising two heavy chains and two light chains, wherein each heavy chain comprises an IgG heavy chain constant region comprising a cysteine at one of the following residues: residue 124 in the C_{H}1 domain, residue 375 in the C_{H}3 domain, and residue 373 in the C_{H}3 domain.
17. The antibody of embodiment 15, wherein said antibody comprises a cysteine at residue 124 in the C_{H}1 domain of each heavy chain and further comprises a cysteine at one, but not all, of residues 375 and 378 in the C_{H}3 domain, and residue 157 in the C_{H}1 domain, of each heavy chain.
18. The antibody of embodiment 16, wherein said antibody comprises a cysteine at residue 375 in the C_{H}3 domain of each heavy chain.
19. The antibody of embodiment 16, wherein said antibody comprises a cysteine at residue 378 in the C_{H}3 domain of each heavy chain.
20. An antibody of any one of embodiments 15 to 18 wherein each of said IgG heavy chain constant regions is a human, mouse, rat or rabbit IgG constant region.
21. The antibody of embodiment 19 wherein each of said IgG heavy chain constant regions is human IgG1 or human IgG4 isotype.
22. The antibody of embodiment 20 wherein each of said IgG heavy chain constant regions is a human IgG1.
23. The antibody of embodiment 15 wherein each of said heavy chain constant regions is human IgG1 given by the amino acid sequence of SEQ ID NO: 17, 18, 19, or 52.
24. The antibody of embodiment 16 wherein each of said heavy chain constant regions is human IgG1 given by the amino acid sequence of SEQ ID NO: 20, 21, or 53.
25. An antibody according to anyone of embodiments 21 to 23 wherein said each of said IgG1 heavy chain constant regions further comprises an isoleucine substituted at residue 247, a glutamine substituted at residue 339, and optionally a glutamic acid substituted at residue 332.
26. The antibody of embodiment 20 wherein each of said IgG heavy chain constant regions is a human IgG4.
27. The antibody of embodiment 15 wherein each of said heavy chain constant regions is human IgG4 given by the amino acid sequence of SEQ ID NO: 12, 13, 14, 54, or 55.
28. The antibody of embodiment 16 wherein each of said heavy chain constant region is human IgG4 given by the amino acid sequence of SEQ ID NO: 15, 16, 56, or 57.
29. An antibody according to anyone of embodiments 25 to 27 wherein each of said IgG4 heavy chain constant region further comprises a proline substituted at residue 228, an alanine substituted at residue 234, and an alanine substituted at residue 235 and a glutamine substituted at residue 339.
30. An antibody according to any one of embodiments 1-28 wherein each cysteine at residue 124, 157, 162, 375 or 378 of each IgG constant region is conjugated to an N-formyl-methionine peptide via a maleimide-PEG linker.
31. The conjugated antibody of embodiment 29 comprising a cysteine at residue 124 of each IgG constant region and a cysteine at one, but not all, of residues 157, 162, 375, and 378 of each IgG constant region, wherein each cysteine at residue 124 and 157, 162, 375, or 378 of each IgG constant region is conjugated to an N-formyl-methionine peptide via a maleimide-PEG linker of the formula

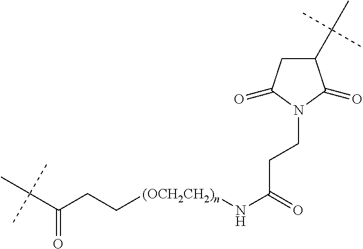

wherein said linker is covalently attached to said antibody through a thioether bond to the cysteine at residue 124 and 157, 162, 375, or 378 of the IgG constant region, and to said N-formyl-methionine peptide through an amide bond at the epsilon amino group of the C-terminal lysine of peptide; and wherein n=6-24.
32. The conjugated antibody of embodiment 30 wherein the cysteine at residue124 and the cysteine at residue 375 of each IgG constant region is conjugated to said N-formyl methionine peptide via said maleimide-PEG linker.
33. The conjugated antibody of embodiment 30 wherein the cysteine at residue124 and the cysteine at residue 378 of each IgG constant region is conjugated to said N-formyl methionine peptide via said maleimide-PEG linker.
34. A conjugated antibody of any one of embodiments 30 to 32 wherein n=12.
35. A conjugated antibody of any one of embodiments 29 to 33, wherein the N-formyl methionine peptide is given by SEQ ID NO: 22, SEQ ID NO: 23, SEQ ID NO: 36, SEQ ID NO: 37, SEQ ID NO: 38, SEQ ID NO: 39, SEQ ID NO: 40, or SEQ ID NO: 41.
36. A pharmaceutical composition comprising a conjugated antibody of any one of embodiments 29 to 34 and one or more pharmaceutically acceptable carriers, diluents or excipients.
37. A method of treating solid cancers or liquid tumors comprising administering to a patient in need thereof an effective amount of a conjugated antibody, or a pharmaceutical composition thereof, according to any one of embodiments 29 to 35.
38. The method according to embodiment 36 for treating breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.
39. A conjugated antibody of any one of embodiments 29 to 35 for use in therapy.
40. A conjugated antibody of any one of embodiments 29 to 35 for use in the treatment of solid cancers or liquid tumors.
41. The conjugated antibody of embodiment 39 for use in the treatment of breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.
42. A compound that is an antibody containing at least one engineered cysteine, wherein the antibody is conjugated by a linker to a chemoattractant that is capable of attracting and/or activating one or more cells of the immune system, and wherein the chemoattractant is conjugated to the antibody at one or more cysteine residues within the antibody.
43. The compound of embodiment 42, wherein the antibody is a monoclonal antibody or a bispecific antibody.
44. The compound of embodiment 42, wherein the antibody is a monoclonal antibody.
45. The compound of embodiment 42, wherein the antibody is a bispecific antibody.
46. The compound of any one of embodiments 42-45, wherein the cysteine is an engineered cysteine within the antibody variable region.
47. The compound of any one of embodiments 42-45, wherein the cysteine is an engineered cysteine within the antibody constant region.
48. The compound of any one of embodiments 42-45, wherein the cysteine is an engineered cysteine within the C_{H}1 or C_{H}3 domains.
49. The compound of any one of embodiments 42-48, wherein the cysteine is engineered at a position to replace a native serine, valine, alanine, glutamine, asparagine, threonine, or glycine.
50. The compound of embodiment 49, wherein the cysteine is engineered at a position to replace a native serine, valine, or alanine.
51. The compound of any one of embodiments 42-50, wherein the total number of engineered cysteines is between two and six.
52. The compound of any one of embodiments 42-51, wherein the compound is capable of attracting and activating one or more cells of the immune system.
53. The compound of any one of embodiments 42-52, wherein the immune system is the adaptive immune system.
54. The compound of any one of embodiments 42-52, wherein the immune system is the innate immune system.
55. The compound of any one of embodiments 42-52, wherein the one of more cells of the immune system are neutrophils.
56. The compound of any one of embodiments 42-52, wherein the one of more cells of the immune system are macrophages.
57. The compound of any one of embodiments 42-56, wherein the linker is a PEG linker or a Mal-Dap linker.
58. The compound of embodiment 57, wherein the linker is a PEG linker.
59. The compound of embodiment 57, wherein the linker is a Mal-Dap linker.
60. The compound of any one of embodiments 42-58, wherein the antibody comprises an IgG heavy chain constant region and a light chain constant region, wherein said constant region comprises an engineered cysteine at at least one of the following residues: residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain.
61. The compound of embodiment 60, wherein said antibody comprises a cysteine at residue 124 in the C_{H}1 domain and further comprises a cysteine at one, but not all, of residue 157 and 162 in the C_{H}1 domain and residues 375 and 378 in the CH3 domain.
62. The compound of embodiment 61, wherein said antibody comprises a cysteine at residue 157 in the CH1 domain.
63. The compound of embodiment 61, wherein said antibody comprises a cysteine at residue 375 in the CH3 domain.
64. The compound of embodiment 61, wherein said antibody comprises a cysteine at residue 378 in the CH3 domain.
65. The compound of any one of embodiments 42-64, wherein said IgG heavy chain constant region is a human, mouse, rat, or rabbit IgG constant region.
66. The compound of embodiment 65, wherein said IgG heavy chain constant region is a human IgG1 or human IgG4 isotype.
67. The compound of embodiment 66, wherein said IgG heavy chain constant region is a human IgG1.
68. The compound of embodiment 67, wherein the heavy chain constant region is human IgG1 given by the amino acid sequence of SEQ ID NO: 17, 18, 19, or 52.
69. The compound of embodiment 67, wherein the heavy chain constant region is human IgG1 given by the amino acid sequence of SEQ ID NO: 20, 21, or 53.
70. The compound of any one of embodiments 66-69, wherein said IgG1 heavy chain constant region further comprises an isoleucine substituted at residue 247, a glutamine substituted at residue 339, and optionally a glutamic acid substituted at residue 332.
71. The compound of embodiment 66, wherein said IgG heavy chain constant region is a human IgG4.
72. The compound of embodiment 71, wherein the heavy chain constant region is human IgG4 given by the amino acid sequence of SEQ ID NO: 12, 13, 14, 54, or 55.
73. The compound of embodiment 71, wherein the heavy chain constant region is human IgG4 given by the amino acid sequence of SEQ ID NO: 15, 16, 56, or 57.
74. An antibody according to any one of embodiments 71-73, wherein said IgG4 heavy chain constant region further comprises a proline substituted at residue 228, an alanine substituted at residue 234, and an alanine substituted at residue 235 and a glutamine substituted at residue 339.
75. The compound of any one of embodiments 42-74, wherein the chemoattractant is a f-Met peptide, small molecule FPR-1 agonists, PRR agonist, peptide mimetics, N-ureido-peptide, or bacterial sugar.
76. The compound of embodiment 75, wherein the chemoattractant is an N-formyl methionine peptide.
77. The compound of embodiment 76, wherein the N-formyl peptide is given by SEQ ID NO: 22, SEQ ID NO: 23, SEQ ID NO: 36, SEQ ID NO: 37, SEQ ID NO: 38, SEQ ID NO: 39, SEQ ID NO: 40, or SEQ ID NO: 41.
78. The compound of any one of embodiments 42-78, wherein the cysteine is conjugated to a chemoattractant via a maleimide-PEG linker.
79. The compound of embodiment 78 wherein the cysteine is conjugated to a chemoattractant via a maleimide-PEG linker of the formula

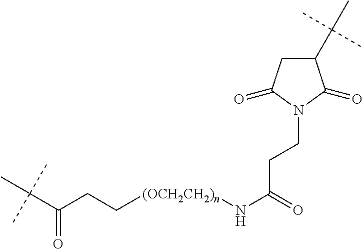

wherein said linker is covalently attached to said antibody through a thioether bond to the cysteine, and to said chemoattractant through an amide bond at the epsilon amino group of the C-terminal lysine of peptide; and wherein n=2-24.
80. The compound of embodiment 79, wherein n=12.
81. A pharmaceutical composition comprising the compound of any one of embodiments 42-80 and one or more pharmaceutically acceptable carriers, diluents or excipients.
82. A method of treating solid cancers or liquid tumors comprising administering to a patient in need thereof an effective amount of a compound, or a pharmaceutical composition thereof, according to any one of embodiments 42-81.
83. The method according to embodiment 82 for treating breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.
84. The compound of any one of embodiments 42-80 for use in therapy.
85. The compound of any one of embodiments 42-80 for use in the treatment of solid cancers or liquid tumors.
86. The compound of any one of embodiments 42-80 for use in the treatment of breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.
87. The compound R—P₁—P₂—P₃—NH(CH₂CH₂O)ₙCH₂CH₂—Y, wherein:
   (i) R is a HC(═O)— or R¹NHC(═O)NH—;
   (ii) R¹ is C₅-C₁₀ aryl which may be substituted or unsubstituted;
   (iii) P₁ is Met or Nle;
   (iv) P₂ is a peptide or peptide mimetic;
   (v) P₃ is Lysine with epsilon amino acylation;
   (vi) n is an integer of from 6-24;
   (vii) Y is maleimide, maleimide-diaminopropionic, iodoacetamide or vinyl sulfone;
   (viii) or a salt thereof.
88. The compound R—P₁—P₂—NH(CH₂CH₂O)ₙCH₂CH₂—P₃—Y, wherein:
   (i) R is a HC(═O)— or R¹NHC(═O)NH—;
   (ii) R¹ is C₅-C₁₀ aryl which may be substituted or unsubstituted;
   (iii) P₁ is Met or Nle;
   (iv) P₂is a peptide or peptide mimetic;
   (v) P₃ is Lysine with epsilon amino acylation;
   (vi) n is an integer of from 6-24;
   (vii) Y is maleimide, maleimide-diaminopropionic, iodoacetamide or vinyl sulfone;
   (viii) or a salt thereof.
89. The compound R-Met-P₂—NH(CH₂CH₂O)ₙCH₂CH₂—X₅—Y, wherein:
   (i) R is a HC(═O)— or R¹NHC(═O)NH—;
   (ii) R¹ is phenyl, 4-chlorophenyl, 4-methoxylphenyl, p-tolyl, m-tolyl, aryl, substituted aryl, or 2-allyl;
   (iii) P₂ is a peptide or peptide mimetic;
   (iv) X₅ is a C₂-C₁₀ diaminoakyl; and
   (v) Y is maleimide, maleimide-diaminopropionic, iodoacetamide or vinyl sulfone;
   (xi) or a salt thereof.
90. The compound [R—P₁—P₂—NH(CH₂CH₂O)ₙCH₂CH₂—]₂-Q-X-Y, wherein:
   (i) R is a HC(═O)— or R¹NHC(═O)NH—;
   (ii) R¹ is C₅-C₁₀ aryl which may be substituted or unsubstituted;
   (iii) P₁ is Met or Nle;
   (iv) P₂ is a peptide or peptide mimetic;
   (v) n is an integer of from 6-24;
   (vi) Q is Lys, Orn, Dap, Dab or other amino bifunctional residue capable of being acylated at alpha amino group and side chain amino group;
   (vii) X is a C₂-C₁₀ diaminoakyl; and
   (viii) Y is maleimide, maleimide-diaminopropionic, iodoacetamide or vinyl sulfone;
   (ix) or a salt thereof.
91. The compound [[R—P₁—P₂—NH(CH₂CH₂O)ₙCH₂CH₂—]₄-(Q)₂-Q-X-Y, wherein:
   (i) R is a HC(═O)— or R¹NHC(═O)NH—;
   (ii) R¹ is C₅-C₁₀ aryl which may be substituted or unsubstituted;
   (iii) P₁ is Met or Nle;
   (iv) P₂ is a peptide or peptide mimetic;
   (v) n is an integer of from 6-24;
   (vi) Q is Lys, Orn, Dap, Dab or other amino bifunctional residue capable of being acylated at alpha amino group and side chain amino group
   (vii) X is a C₂-C₁₀ diaminoakyl; and
   (viii) Y is maleimide, maleimide-diaminopropionic, iodoacetamide or vinyl sulfone;
   (ix) or a salt thereof.
92. The compound [[[R—P₁—P₂—NH(CH₂CH₂O)ₙCH₂CH₂]₈-(Q)₄-(Q)₂-Q-X-Y, wherein:
   (i) R is a HC(═O)— or R¹NHC(═O)NH—;
   (ii) R¹ is C₅-C₁₀ aryl which may be substituted or unsubstituted;
   (iii) P₁ is Met or Nle;
   (iv) P₂ is a peptide or peptide mimetic;
   (v) n is an integer of from 6-24;
   (vi) Q is Lys, Orn, Dap, Dab or other amino bifunctional residue capable of being acylated at alpha amino group and side chain amino group
   (vii) X is a C₂-C₁₀ diaminoakyl; and
   (viii) Y is maleimide, maleimide-diaminopropionic, iodoacetamide or vinyl sulfone;
   (ix) or a salt thereof.
93. The compound of any one of embodiments 87-92, wherein P₂ is given by X₁—X₂—X₃—X₄, and wherein:
   (i) X₁ is Leu, Ile, Nle, diethylglycine, or dipropylglcyine;
   (ii) X₂ is Phe, α-Me-Phe, DPhe, 4-F-Phe, 2-Nal, or 1-Nal;
   (iii) X₃ is Glu, Leu, Nle, α-Me-Leu, DLeu, or absent; and
   (iv) X₄ is Glu, DGlu, γGlu, Gla, or absent.
94. The compound of any one of embodiments 87-93, wherein the compound is capable of covalent attachment to an antibody or antibody fragment through a thioether bond.
95. The compound of any one of embodiments 87-94, wherein the compound is capable of covalent attachment to an antibody or antibody fragment through a thioether bond at cysteine residue 124 in the C_{H}1 domain, residue 157 in the C_{H}1 domain, residue 162 in the C_{H}1 domain, residue 262 in the C_{H}2 domain, residue 375 in the C_{H}3 domain, residue 373 in the C_{H}3 domain, residue 397 in the C_{H}3 domain, residue 415 in the C_{H}3 domain, residue 156 in the Cₖₐₚₚₐ domain, residue 171 in the Cₖₐₚₚₐ domain, residue 191 in the Cₖₐₚₚₐ domain, residue 193 in the Cₖₐₚₚₐ domain, residue 202 in the Cₖₐₚₚₐ domain, or residue 208 in the Cₖₐₚₚₐ domain.
96. A compound that is an antibody containing at least one cysteine conjugated by a linker to the compound of any one of embodiments 87-95, that is capable of attracting and/or activating one or more cells of the immune system, and wherein the agent is conjugated to the antibody at one or more cysteine residues within the antibody.

SEQUENCES

Antibody Heavy Chain of Emibetuzumab 378C Conjugates
(SEQ ID NO: 1)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIXVEWESNGQPENNYKTTPPVLDSDGSFFLYS
RLTVDKSRWQEGNVFSCSVMHEALHNHYTQKSLSLSLG
(X at position 373 is cysteine residue modified by thioether bond formation
to maleimide-PEG linker)
Antibody Heavy Chain of Emibetuzumab 124C Conjugates
(SEQ ID NO: 2)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPXVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLG
(X at position 122 is cysteine residue modified by thioether bond formation
to maleimide-PEG linker)
Antibody Heavy Chain of Emibetuzumab 124C-378C Conjugates
(SEQ ID NO: 3)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPXVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIXVEWESNGQPENNYKTTPPVLDSDGSFFLYS
RLTVDKSRWQEGNVFSCSVMHEALHNHYTQKSLSLSLG
(X at position 122 and X at position 373 is cysteine residue modified by
thioether bond formation to maleimide-PEG linker)
Antibody Heavy Chain of Emibetuzumab 124C-375C Conjugates
(SEQ ID NO: 4)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPXVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPXDIAVEWESNGQPENNYKTTPPVLDSDGSFFLYS
RLTVDKSRWQEGNVFSCSVMHEALHNHYTQKSLSLSLG
(X at position 122 and X at position 370 is cysteine residue modified by
thioether bond formation to maleimide-PEG linker)
Antibody Light Chain of Emibetuzumab Conjugates
(SEQ ID NO: 5)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Antibody Heavy Chain of TMab 124C-378C Conjugates
(SEQ ID NO: 6)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGTL
VTVSSASTKGPXVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSRDELTKNQVSLTCLVKGFYPSDIXVEWESNGQPENNYKTTPPVLDSDGSF
FLYSKLTVDKSRWQQGNVFSCSVMHEALHNHYTQKSLSLSPG
(X at position 127 and X at position 381 is cysteine residue modified by
thioether bond formation to maleimide-PEG linker)
Antibody Light Chain of TMab Conjugates
(SEQ ID NO: 7)
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Antibody Heavy Chain of AME133 124C-378C Conjugates
(SEQ ID NO: 8)
EVQLVQSGAEVKKPGESLKISCKGSGRTFTSYNMHWVRQMPGKGLEWMGAIYPLTGD
TSYNQKSKLQVTISADKSISTAYLQWSSLKASDTAMYYCARSTYVGGDWQFDVWGKG
TTVTVSSASTKGPXVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCP
PCPAPELLGGPSVFLFPPKIKDTLMISRTPEVTCVVVDVSHEDPEVKFNWYVDGVEVHN
AKTKPREEQYNSTYRVVSVLTVLHQDWLNGKEYKCKVSNKALPAPIEKTISKQKGQPRE
PQVYTLPPSRDELTKNQVSLTCLVKGFYPSDIXVEWESNGQPENNYKTTPPVLDSDGS
FFLYSKLTVDKSRWQQGNVFSCSVMHEALHNHYTQKSLSLSPG
(X at position 128 and X at position 382 is cysteine residue modified by
thioether bond formation to maleimide-PEG linker)
Antibody Light Chain of AME133 Conjugates
(SEQ ID NO: 9)
EIVLTQSPGTLSLSPGERATLSCRASSSVPYIHWYQQKPGQAPRLLIYATSALASGIPDR
FSGSGSGTDFTLTISRLEPEDFAVYYCQQWLSNPPTFGQGTKLEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
Human IgG1 Constant Region
(SEQ ID NO: 10)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
Human IgG4 Constant Region
(SEQ ID NO: 11)
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLG
Antibody Heavy Chain Constant Region of IgG4 124C Conjugates
(SEQ ID NO: 12)
ASTKGPXVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLG
(X at position 7 is cysteine residue modified by thioether bond formation to
maleimide-PEG linker)
Antibody Heavy Chain Constant Region of IgG4 378C Conjugates
(SEQ ID NO: 13)
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIXVEWESNGQPENNYKTTPPVLDSDGSFFLYSRLT
VDKSRWQEGNVFSCSVMHEALHNHYTQKSLSLSLG
(X at position 258 is cysteine residue modified by thioether bond formation
to maleimide-PEG linker)
Antibody Heavy Chain Constant Region of IgG4 375C Conjugates
(SEQ ID NO: 14)
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPXDIAVEWESNGQPENNYKTTPPVLDSDGSFFLYSRLT
VDKSRWQEGNVFSCSVMHEALHNHYTQKSLSLSLG
(X at position 255 is cysteine residue modified by thioether bond formation
to maleimide-PEG linker)
Antibody Heavy Chain Constant Region of IgG4 124C-378C Conjugates
(SEQ ID NO: 15)
ASTKGPXVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIXVEWESNGQPENNYKTTPPVLDSDGSFFLYSRLT
VDKSRWQEGNVFSCSVMHEALHNHYTQKSLSLSLG
(X at position 7 and X at position 258 is cysteine residue modified by
thioether bond formation to maleimide-PEG linker)
Antibody Heavy Chain Constant Region of IgG4 124C-375C Conjugates
(SEQ ID NO: 16)
ASTKGPXVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPXDIAVEWESNGQPENNYKTTPPVLDSDGSFFLYSRLT
VDKSRWQEGNVFSCSVMHEALHNHYTQKSLSLSLG
(X at position 7 and X at position 255 is cysteine residue modified by
thioether bond formation to maleimide-PEG linker)
Antibody Heavy Chain Constant Region of IgG1 124C Conjugates
(SEQ ID NO: 17)
ASTKGPXVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
(X at position 7 is cysteine residue modified by thioether bond formation to
maleimide-PEG linker)
Antibody Heavy Chain Constant Region of IgG1 378C Conjugates
(SEQ ID NO: 18)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIXVEWESNGQPENNYKTTPPVLDSDGSFFLYSK
LTVDKSRWQQGNVFSCSVMHEALHNHYTQKSLSLSPGK
(X at position 261 is cysteine residue modified by thioether bond formation
to maleimide-PEG linker)
Antibody Heavy Chain Constant Region of IgG1 375C Conjugates
(SEQ ID NO: 19)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPXDIAVEWESNGQPENNYKTTPPVLDSDGSFFLYSK
LTVDKSRWQQGNVFSCSVMHEALHNHYTQKSLSLSPGK
(X at position 258 is cysteine residue modified by thioether bond formation
to maleimide-PEG linker)
Antibody Heavy Chain Constant Region of IgG1 124C-378C Conjugates
(SEQ ID NO: 20)
ASTKGPXVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIXVEWESNGQPENNYKTTPPVLDSDGSFFLYSK
LTVDKSRWQQGNVFSCSVMHEALHNHYTQKSLSLSPGK
(X at position 7 and X at position 261 is cysteine residue modified by
thioether bond formation to maleimide-PEG linker)
Antibody Heavy Chain Constant Region of IgG1 124C-375C Conjugates
(SEQ ID NO: 21)
ASTKGPXVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPXDIAVEWESNGQPENNYKTTPPVLDSDGSFFLYSK
LTVDKSRWQQGNVFSCSVMHEALHNHYTQKSLSLSPGK
(X at position 7 and X at position 258 is cysteine residue modified by
thioether bond formation to maleimide-PEG linker)
fMLFX (Peptide-'183)
(SEQ ID NO: 22)
(Met at position 1 is formylated)
(X at position 4 is lysine residue modified by amide bond formation to
maleimide-PEG linker)
fMLFK
(SEQ ID NO: 23)
(Met at position 1 is formylated)
MLFX (Peptide-'844)
(SEQ ID NO: 24)
(X at position 4 is lysine residue modified by amide bond formation to
maleimide-PEG linker)
(SEQ ID NO: 25)
MLFK
Antibody Heavy Chain of MET415C Antibody Conjugates
(SEQ ID NO: 26)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKXRWQEGNVFSCSVMHEALHNHYTQKSLSLSLG
(X at position 410 is cysteine residue modified by thioether bond formation to
maleimide-PEG linker)
Antibody Light Chain of MET156C Antibody Conjugates
(SEQ ID NO: 27)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQXGNSQESVTEQDSKDSTYSLS
STLTLSKADYEKHKVYACEVTHQGLSSPVTKSFNRGEC
(X at position 157 is cysteine residue modified by thioether bond formation to
maleimide-PEG linker)
Antibody Light Chain of MET171C Antibody Conjugates
(SEQ ID NO: 28)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDXTYSLS
STLTLSKADYEKHKVYACEVTHQGLSSPVTKSFNRGEC
(X at position 172 is cysteine residue modified by thioether bond formation to
maleimide-PEG linker)
Antibody Light Chain of MET191C Antibody Conjugates
(SEQ ID NO: 29)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDSTYSLS
STLTLSKADYEKHKXYACEVTHQGLSSPVTKSFNRGEC
(X at position 192 is cysteine residue modified by thioether bond formation to
maleimide-PEG linker)
Antibody Light Chain of MET193C Antibody Conjugates
(SEQ ID NO: 30)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDSTYSLS
STLTLSKADYEKHKVYXCEVTHQGLSSPVTKSFNRGEC
(X at position 194 is cysteine residue modified by thioether bond formation to
maleimide-PEG linker)
Antibody Light Chain of MET202C Antibody Conjugates
(SEQ ID NO: 31)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLXSPVTKSFNRGEC
(X at position 203 is cysteine residue modified by thioether bond formation to
maleimide-PEG linker)
Antibody Light Chain of MET208C Antibody Conjugates
(SEQ ID NO: 32)
DIQMTQSPSSLSASVGDRVTITCSVSSSVSSIYLHWYQQKPGKAPKLLIYSTSNLASGVP
SRFSGSGSGTDFTLTISSLQPEDFATYYCQVYSGYPLTFGGGTKVEIKRTVAAPSVFIFP
PSDEQLKSGTASVVCLLNNFYPREAKVQWKVDNALQSGNSQESVTEQDSKDSTYSLS
STLTLSKADYEKHKVYACEVTHQGLSSPVTKXFNRGEC
(X at position 209 is cysteine residue modified by thioether bond formation to
maleimide-PEG linker)
Antibody Heavy Chain of Trastuzumab 124C-157C Antibody Conjugates
(SEQ ID NO: 33)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGTL
VTVSSASTKGPXVFPLAPSSKSTSGGTAALGCLVKDYFPEPVTVXWNSGALTSGVHTF
PAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPG
(X at position 127 and X at position 160 is cysteine residue modified by thioether
bond formation to maleimide-PEG linker)
Antibody Heavy Chain Aof 124C-378C Bispecific Antibody I Conjugate
(SEQ ID NO: 34)
EVQLVESGGGLVQPGGSLRLSCAASGFTFTDYTMDWVRKAPGKGLEWVADVNPNSG
GSIYNQEFKGRFTLSVDRSKNTLYLQMNSLRAEDTAVYYCARNLGPSFYFDYWGQGTL
VTVSSASTKGPXVFPLAPSSKSTSGGTAALGCLVKDYFPEPVTVSWNSGALTSGVATG
PAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNA
KTKPREEQYQSTYRVVSVLTVLHQDWLNGKEYKCKVSNKALPAPIEKTISKAKGQPREP
QVYTLPPSRDELTKNQVSLTCLVKGFYPSDIXVEWESNGQPENNYDTTPPVLDSDGSF
FLYSDLTVDKSRWQQGNVFSCSVMHEALHNHYTQKSLSLSPG
(X at position 126 and X at position 380 is cysteine residue modified by thioether
bond formation to maleimide-PEG linker)
Antibody Heavy Chain B of 124C-378C Bispecific Antibody I Conjugate
(SEQ ID NO: 35)
QVQLVQSGAEVKKPGASVKVSCKASGYTFTSHWMHWVRYAPGQGLEWIGEF
NPSNGRTNYNEKFKSKATMTVDTSTNTAYMELSSLRSEDTAVYYCASRDYDYD
GRYFDYWGQGTLVTVSSASTKGPXVFPLAPSSKSTSGGTAALGCLVKDYFPEP
VTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTC
VVVDVSHEDPEVKFNWYVDGVEVHNAKTKPREEQYQSTYRVVSVLTVLHQDW
LNGKEYKCKVSNKALPAPIEKTISKAKGQPREPQVYTLPPSRKELTKNQVSLTCL
VKGFYPSDIXVEWESNGQPENNYKTTPPVLKSDGSFFLYSKLTVDKSRWQQGN
VFSCSVMHEALHNHYTQKSLSLSPG
(X at position 128 and X at position 382 is cysteine residue modified by thioether
bond formation to maleimide-PEG linker)
fMIFLX (FRM-021)
(SEQ ID NO: 36)
(Met at position 1 is formylated)
(X at position 5 is lysine residue side chain modified through epsilon amide bond
formation to a hydrolyzed maleimide-PEG linker)
fMXFX (FRM-029)
(SEQ ID NO: 37)
(Met at position 1 is formylated)
(X at position 2 is diethylglycine)
(X at position 4 is leucine residue C-terminally connected by amide bond
formation to a PEG linker of the formula (PEG6)₂-NH—(CH₂)₂—NH₂)
fMXFX (FRM-030)
(SEQ ID NO: 38)
(Met at position 1 is formylated)
(X at position 2 is dipropylglycine)
(X at position 4 is leucine residue C-terminally connected by amide bond
formation to a PEG linker of the formula (PEG6)₂—NH—(CH₂)₂—NH₂)
fMIX (FRM-031)
(SEQ ID NO: 39)
(Met at position 1 is formylated)
(X at position 3 is phenylalanine residue C-terminally connected by amide bond
formation to a PEG linker of the formula PEG12-NH—(CH₂)₂—NH₂)
fMIFX (FRM-023)
(SEQ ID NO: 40)
(Met at position 1 is formylated)
(X at position 4 is leucine residue C-terminally connected by amide bond
formation to a PEG linker of the formula PEG12-NH—(CH₂)—NH₂)
fMIFX (FRM-032)
(SEQ ID NO: 41)
(Met at position 1 is formylated)
(X at position 4 is leucine residue modified by amide bond formation to a linker of
the formula NH—(CH₂)—NH-[(Mal-Dap(NH₂)])
fNIeLX (FRM-009)
(SEQ ID NO: 42)
(Nle at position 1 is formylated)
(X at position 3 is phenylalanine C-terminally connected by amide bond formation
to a linker of the formula PEG12-Lys(Maleimido-Propionyl)-OH)
Antibody Heavy Chain of Emibetuzumab Conjugates
(SEQ ID NO: 43)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLG
Antibody Heavy Chain of Emibetuzumab 157C Antibody Conjugates
(SEQ ID NO: 44)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVXWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLG
(X at position 155 is cysteine residue modified by thioether bond formation to
maleimide-PEG linker)
Antibody Heavy Chain of Emibetuzumab 162C Antibody Conjugates
(SEQ ID NO: 45)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGXLTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLG
(X at position 160 is cysteine residue modified by thioether bond formation to
maleimide-PEG linker)
Antibody Heavy Chain of Emibetuzumab 262C Antibody Conjugates
(SEQ ID NO: 46)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCXVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYS
RLTVDKSRWQEGNVFSCSVMHEALHNHYTQKSLSLSLG
(X at position 257 is cysteine residue modified by thioether bond formation to
maleimide-PEG linker)
Antibody Heavy Chain of Emibetuzumab 375C Antibody Conjugates
(SEQ ID NO: 47)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPXDIAVEWESNGQPENNYKTTPPVLDSDGSFFLYS
RLTVDKSRWQEGNVFSCSVMHEALHNHYTQKSLSLSLG
(X at position 370 is cysteine residue modified by thioether bond formation to
maleimide-PEG linker)
Antibody Heavy Chain of Emibetuzumab 397C Antibody Conjugates
(SEQ ID NO: 48)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPXLDSDGSFFLYS
RLTVDKSRWQEGNVFSCSVMHEALHNHYTQKSLSLSLG
(X at position 392 is cysteine residue modified by thioether bond formation to
maleimide-PEG linker)
Antibody Heavy Chain of Emibetuzumab 124C-157C-378C Antibody Conjugates
(SEQ ID NO: 49)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPXVFPLAPCSRSTSESTAALGCLVKDYFPEPVTVXWNSGALTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIXVEWESNGQPENNYKTTPPVLDSDGSFFLYS
RLTVDKSRWQEGNVFSCSVMHEALHNHYTQKSLSLSLG
(X at position 122 and X at position 155 and X at position 373 is cysteine residue
modified by thioether bond formation to maleimide-PEG linker)
Antibody Heavy Chain of Emibetuzumab 124C-162C-378C Antibody Conjugates
(SEQ ID NO: 50)
QVQLVQSGAEVKKPGASVKVSCKASGYTFTDYYMHWVRQAPGQGLEWMGRVNPNR
RGTTYNQKFEGRVTMTTDTSTSTAYMELRSLRSDDTAVYYCARANWLDYWGQGTTVT
VSSASTKGPXVFPLAPCSRSTSESTAALGCLVKDYFPEPVTVSWNSGXLTSGVHTFPA
VLQSSGLYSLSSVVTVPSSSLGTKTYTCNVDHKPSNTKVDKRVESKYGPPCPPCPAPE
AAGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYT
LPPSQEEMTKNQVSLTCLVKGFYPSDIXVEWESNGQPENNYKTTPPVLDSDGSFFLYS
RLTVDKSRWQEGNVFSCSVMHEALHNHYTQKSLSLSLG
(X at position 122 and X at position 160 and X at position 373 is cysteine residue
modified by thioether bond formation to maleimide-PEG linker)
Antibody Heavy Chain of Tmab (IQE) 124C-378C Antibody Conjugates
(SEQ ID NO: 51)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGTL
VTVSSASTKGPXVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPC
PAPELLGGPSVFLFPPKIKDTLMISRTPEVTCVVVDVSHEDPEVKFNWYVDGVEVHNAK
TKPREEQYNSTYRVVSVLTVLHQDWLNGKEYKCKVSNKALPAPEEKTISKQKGQPREP
QVYTLPPSRDELTKNQVSLTCLVKGFYPSDIXVEWESNGQPENNYKTTPPVLDSDGSF
FLYSKLTVDKSRWQQGNVFSCSVMHEALHNHYTQKSLSLSPG
(X at position 127 and X at position 381 is cysteine residue modified by thioether
bond formation to maleimide-PEG linker)
Antibody Heavy Chain Constant Region of IgG1 157C Conjugates
(SEQ ID NO: 52)
ASTKGPSVFPLAPSSKSTSGGTAALGCLVKDYFPEPVTVXWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPG
(X at position 40 is cysteine residue modified by thioether bond formation to
maleimide-PEG linker)
Antibody Heavy Chain Constant Region of IgG1 124C-157C Conjugates
(SEQ ID NO: 53)
ASTKGPXVFPLAPSSKSTSGGTAALGCLVKDYFPEPVTVXWNSGALTSGVHTFPAVLQ
SSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPG
(X at position 7 and X at position 40 is cysteine residue modified by thioether
bond formation to maleimide-PEG linker)
Antibody Heavy Chain Constant Region of IgG4 157C Conjugates
(SEQ ID NO: 54)
ASTKGPSVFPLAPCSRSTSESTAALGCLVKDYFPEPVTVXWNSGALTSGVHTFPAVLQ
SSGLYSLSSVVTVPSSSLGTKTYTCNVDHKPSNTKVDKRVESKYGPPCPPCPAPEAAG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLG
(X at position 40 is cysteine residue modified by thioether bond formation to
maleimide-PEG linker)
Antibody Heavy Chain Constant Region of IgG4 162C Conjugates
(SEQ ID NO: 55)
ASTKGPSVFPLAPCSRSTSESTAALGCLVKDYFPEPVTVSWNSGXLTSGVHTFPAVLQ
SSGLYSLSSVVTVPSSSLGTKTYTCNVDHKPSNTKVDKRVESKYGPPCPPCPAPEAAG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGSFFLYSRLT
VDKSRWQEGNVFSCSVMHEALHNHYTQKSLSLSLG
(X at position 45 is cysteine residue modified by thioether bond formation to
maleimide-PEG linker)
Antibody Heavy Chain Constant Region of IgG4 124C-157C-373C Conjugates
(SEQ ID NO: 56)
ASTKGPXVFPLAPCSRSTSESTAALGCLVKDYFPEPVTVXWNSGALTSGVHTFPAVLQ
SSGLYSLSSVVTVPSSSLGTKTYTCNVDHKPSNTKVDKRVESKYGPPCPPCPAPEAAG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIXVEWESNGQPENNYKTTPPVLDSDGSFFLYSRLT
VDKSRWQEGNVFSCSVMHEALHNHYTQKSLSLSLG
(X at position 7 and X at position 40 and X at position 258 is cysteine residue
modified by thioether bond formation to maleimide-PEG linker)
Antibody Heavy Chain Constant Region of IgG4 124C-162C-373C Conjugates
(SEQ ID NO: 57)
ASTKGPXVFPLAPCSRSTSESTAALGCLVKDYFPEPVTVSWNSGXLTSGVHTFPAVLQ
SSGLYSLSSVVTVPSSSLGTKTYTCNVDHKPSNTKVDKRVESKYGPPCPPCPAPEAAG
GPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPP
SQEEMTKNQVSLTCLVKGFYPSDIXVEWESNGQPENNYKTTPPVLDSDGSFFLYSRLT
VDKSRWQEGNVFSCSVMHEALHNHYTQKSLSLSLG
(X at position 7 and X at position 45 and X at position 258 is cysteine residue
modified by thioether bond formation to maleimide-PEG linker)
Antibody Light Chain A of Bispecific Antibody I Conjugate
(SEQ ID NO: 58)
RIQMTQSPSSLSASVGDRVTITCKASQDVSIGVAWYQDKPGKAPKLLIYSASYRYTGVP
SRFSGSGSGTDFTLTISSLQPEDFATYYCQQYYIYPYTFGQGTKVEIKGQPKAAPSVTLF
PPSSEELQANKATLVCYISDFYPGAVTVAWKADSSPVKAGVETTTPSKQSNNKYAAWS
YLSLTPEQWKSHRSYSCQVTHEGSTVEKTVAPTEC
Antibody Light Chain B of Bispecific Antibody I Conjugate
(SEQ ID NO: 59)
DIQMTQSPSSLSASVGDRVTITCSASSSVTYMYWYQRKPGKAPKLLIYDTSNLASGVPS
RFSGSGSGTDYTFTISSLQPEDIATYYCQQWSSHIFTFGQGTKVEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSFNRGEC

We claim:

1. An antibody or antibody fragment comprising a human IgG4 heavy chain constant region comprising two engineered cysteines at residues 124 and 378 (according to EU Index numbering).

2. The antibody or antibody fragment of claim 1, wherein the antibody or antibody fragment comprises SEQ ID NO: 15.

3. A conjugate comprising the antibody or antibody fragment of claim 1.

4. A conjugate comprising the antibody or antibody fragment of claim 2.

5. An antibody or antibody fragment comprising a human IgG1 heavy chain constant region comprising two engineered cysteines at residues 124 and 378 (according to EU Index numbering).

6. The antibody or antibody fragment of claim 5, wherein the antibody or antibody fragment comprises SEQ ID NO: 20.

7. A conjugate comprising the antibody or antibody fragment of claim 5.

8. A conjugate comprising the antibody or antibody fragment of claim 6.

9. An antibody or antibody fragment comprising a human IgG4 heavy chain constant region comprising a single engineered cysteine at residue 378 (according to EU Index numbering).

10. The antibody or antibody fragment of claim 9, wherein the antibody or antibody fragment comprises SEQ ID NO: 13.

11. A conjugate comprising the antibody or antibody fragment of claim 9.

12. A conjugate comprising the antibody or antibody fragment of claim 10.

13. An antibody or antibody fragment comprising a human IgG4 heavy chain constant region comprising a single engineered cysteine at residue 124 (according to EU Index numbering).

14. The antibody or antibody fragment of claim 13, wherein the antibody or antibody fragment comprises SEQ ID NO: 12.

15. A conjugate comprising the antibody or antibody fragment of claim 13.

16. A conjugate comprising the antibody or antibody fragment of claim 14.

17. An antibody or antibody fragment comprising a human IgG1 heavy chain constant region comprising a single engineered cysteine at residue 378 (according to EU Index numbering).

18. The antibody or antibody fragment of claim 17, wherein the antibody or antibody fragment comprises SEQ ID NO: 18.

19. A conjugate comprising the antibody or antibody fragment of claim 17.

20. A conjugate comprising the antibody or antibody fragment of claim 18.

21. An antibody or antibody fragment comprising a human IgG1 heavy chain constant region comprising a single engineered cysteine at residue 124 (according to EU Index numbering).

22. The antibody or antibody fragment of claim 21, wherein the antibody or antibody fragment comprises SEQ ID NO: 17.

23. A conjugate comprising the antibody or antibody fragment of claim 21.

24. A conjugate comprising the antibody or antibody fragment of claim 22.


SEQUENCE LISTING (59 sequences)

SEQ ID NO: 1 - protein, 441 aa, synthetic construct
  REGION 1..441: note = Synthetic construct
  SITE 373: note = THIOETH - position 373 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 2 - protein, 441 aa, synthetic construct
  REGION 1..441: note = Synthetic construct
  SITE 122: note = THIOETH - position 122 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 441 aa, synthetic construct
  REGION 1..441: note = Synthetic construct
  SITE 122: note = THIOETH - position 122 is cysteine residue modified by thioether bond formation to maleimide-polyethylene glycol (PEG) linker
  SITE 373: note = THIOETH - position 373 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 4 - protein, 441 aa, synthetic construct
  REGION 1..441: note = Synthetic construct
  SITE 122: note = THIOETH - position 122 is cysteine residue modified by thioether bond formation to maleimide-polyethylene glycol (PEG) linker
  SITE 370: note = THIOETH - position 370 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPCDIAVEWESNGQPENNYKTTPPVLDSDGSFFLYSRLTVDKSRWQEGNVFSCSVMHEALHNHYTQKSLSLSLG

SEQ ID NO: 5 - protein, 215 aa, synthetic construct
  REGION 1..215: note = Synthetic construct
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, synthetic construct
  REGION 1..449: note = Synthetic construct
  SITE 127: note = THIOETH - position 127 is cysteine residue modified by thioether bond formation to maleimide-polyethylene glycol (PEG) linker
  SITE 381: note = THIOETH - position 381 is cysteine residue modified by thioether bond formation to maleimide-polyethylene glycol (PEG) linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDICVEWESNGQPENNYKTTPPVLDSDGSFFLYSKLTVDKSRWQQGNVFSCSVMHEALHNHYTQKSLSLSPG

SEQ ID NO: 7 - protein, 214 aa, synthetic construct
  REGION 1..214: note = Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, synthetic construct
  REGION 1..450: note = Synthetic construct
  SITE 128: note = THIOETH - position 128 is cysteine residue modified by thioether bond formation to maleimide-polyethylene glycol (PEG) linker
  SITE 382: note = THIOETH - position 382 is cysteine residue modified by thioether bond formation to maleimide-polyethylene glycol (PEG) linker
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSRDELTKNQVSLTCLVKGFYPSDICVEWESNGQPENNYKTTPPVLDSDGSFFLYSKLTVDKSRWQQGNVFSCSVMHEALHNHYTQKSLSLSPG

SEQ ID NO: 9 - protein, 213 aa, synthetic construct
  REGION 1..213: note = Synthetic construct
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 326 aa, synthetic construct
  REGION 1..326: note = Synthetic construct
  SITE 7: note = THIOETH - position 7 is cysteine residue modified by thioether bond formation to maleimide-polyethylene glycol (PEG) linker
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 326 aa, synthetic construct
  REGION 1..326: note = Synthetic construct
  SITE 258: note = THIOETH - position 258 is cysteine residue modified by thioether bond formation to maleimide-polyethylene glycol (PEG) linker
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 14 - protein, 326 aa, synthetic construct
  REGION 1..326: note = Synthetic construct
  SITE 255: note = THIOETH - position 255 is cysteine residue modified by thioether bond formation to maleimide-polyethylene glycol (PEG) linker
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPCDIAVEWESNGQPENNYKTTPPVLDSDGSFFLYSRLTVDKSRWQEGNVFSCSVMHEALHNHYTQKSLSLSLG

SEQ ID NO: 15 - protein, 326 aa, synthetic construct
  REGION 1..326: note = Synthetic construct
  SITE 7: note = THIOETH - position 7 is cysteine residue modified by thioether bond formation to maleimide-polyethylene glycol (PEG) linker
  SITE 258: note = THIOETH - position 258 is cysteine residue modified by thioether bond formation to maleimide-polyethylene glycol (PEG) linker
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 16 - protein, 326 aa, synthetic construct
  REGION 1..326: note = Synthetic construct
  SITE 7: note = THIOETH - position 7 is cysteine residue modified by thioether bond formation to maleimide-polyethylene glycol (PEG) linker
  SITE 255: note = THIOETH - position 255 is cysteine residue modified by thioether bond formation to maleimide-polyethylene glycol (PEG) linker
ASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPCDIAVEWESNGQPENNYKTTPPVLDSDGSFFLYSRLTVDKSRWQEGNVFSCSVMHEALHNHYTQKSLSLSLG

SEQ ID NO: 17 - protein, 330 aa, synthetic construct
  REGION 1..330: note = Synthetic construct
  SITE 7: note = THIOETH - position 7 is cysteine residue modified by thioether bond formation to maleimide-polyethylene glycol (PEG) linker
ASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, synthetic construct
  REGION 1..330: note = Synthetic construct
  SITE 261: note = THIOETH - position 261 is cysteine residue modified by thioether bond formation to maleimide-polyethylene glycol (PEG) linker
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 19 - protein, 330 aa, synthetic construct
  REGION 1..330: note = Synthetic construct
  SITE 258: note = THIOETH - position 258 is cysteine residue modified by thioether bond formation to maleimide-polyethylene glycol (PEG) linker
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, synthetic construct
  REGION 1..330: note = Synthetic construct
  SITE 7: note = THIOETH - position 7 is cysteine residue modified by thioether bond formation to maleimide-polyethylene glycol (PEG) linker
  SITE 261: note = THIOETH - position 261 is cysteine residue modified by thioether bond formation to maleimide-polyethylene glycol (PEG) linker
ASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 21 - protein, 330 aa, synthetic construct
  REGION 1..330: note = Synthetic construct
  SITE 7: note = THIOETH - position 7 is cysteine residue modified by thioether bond formation to maleimide-polyethylene glycol (PEG) linker
  SITE 258: note = THIOETH - position 258 is cysteine residue modified by thioether bond formation to maleimide-polyethylene glycol (PEG) linker
ASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 22 - protein, 4 aa, synthetic construct
  REGION 1..4: note = Synthetic construct
  SITE 1: note = FORMYLATION - Formylation of residue
  SITE 4: note = MISC_FEATURE - position 4 is lysine residue modified by amide bond formation to maleimide-polyethylene glycol (PEG) linker
MLFK

SEQ ID NO: 23 - protein, 4 aa, synthetic construct
  REGION 1..4: note = Synthetic construct
  SITE 1: note = FORMYLATION - Formylation of residue
MLFK

SEQ ID NO: 24 - protein, 4 aa, synthetic construct
  REGION 1..4: note = Synthetic construct
  SITE 4: note = MISC_FEATURE - position 4 is lysine residue modified by amide bond formation to maleimide-polyethylene glycol (PEG) linker
MLFK

SEQ ID NO: 25 - protein, 4 aa, synthetic construct
  REGION 1..4: note = Synthetic construct
MLFK

SEQ ID NO: 26 - protein, 441 aa, synthetic construct
  REGION 1..441: note = Synthetic Construct
  SITE 410: note = THIOETH - position 410 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKCRWQEGNVFSCSVMHEALHNHYTQKSLSLSLG

SEQ ID NO: 27 - protein, 215 aa, synthetic construct
  REGION 1..215: note = Synthetic Construct
  SITE 157: note = THIOETH - position 157 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQCGNSQESVTEQDSKDSTYSLSSTLTLSKADYEKHKVYACEVTHQGLSSPVTKSFNRGEC

SEQ ID NO: 28 - protein, 215 aa, synthetic construct
  REGION 1..215: note = Synthetic Construct
  SITE 172: note = THIOETH - position 172 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 29 - protein, 215 aa, synthetic construct
  REGION 1..215: note = Synthetic Construct
  SITE 192: note = THIOETH - position 192 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKCYACEVTHQGLSSPVTKSFNRGEC

SEQ ID NO: 30 - protein, 215 aa, synthetic construct
  REGION 1..215: note = Synthetic Construct
  SITE 194: note = THIOETH - position 194 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYCCEVTHQGLSSPVTKSFNRGEC

SEQ ID NO: 31 - protein, 215 aa, synthetic construct
  REGION 1..215: note = Synthetic Construct
  SITE 203: note = THIOETH - position 203 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLCSPVTKSFNRGEC

SEQ ID NO: 32 - protein, 215 aa, synthetic construct
  REGION 1..215: note = Synthetic Construct
  SITE 209: note = THIOETH - position 209 is cysteine residue modified by thioether bond formation to maleimide-polyethylene glycol (PEG) linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKCFNRGEC

SEQ ID NO: 33 - protein, 449 aa, synthetic construct
  REGION 1..449: note = Synthetic Construct
  SITE 127: note = THIOETH - position 127 is cysteine residue modified by thioether bond formation to maleimide-polyethylene glycol (PEG) linker
  SITE 160: note = THIOETH - position 160 is cysteine residue modified by thioether bond formation to maleimide-polyethylene glycol (PEG) linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPCVFPLAPSSKSTSGGTAALGCLVKDYFPEPVTVCWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 448 aa, synthetic construct
  REGION 1..448: note = Synthetic Construct
  SITE 126: note = THIOETH - position 126 is cysteine residue modified by thioether bond formation to maleimide-polyethylene glycol (PEG) linker
  SITE 380: note = THIOETH - position 380 is cysteine residue modified by thioether bond formation to maleimide-polyethylene glycol (PEG) linker
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPCVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDICVEWESNGQPENNYDTTPPVLDSDGSFFLYSDLTVDKSRWQQGNVFSCSVMHEALHNHYTQKSLSLSPG

SEQ ID NO: 35 - protein, 450 aa, synthetic construct
  REGION 1..450: note = Synthetic Construct
  SITE 128: note = THIOETH - position 128 is cysteine residue modified by thioether bond formation to maleimide-polyethylene glycol (PEG) linker
  SITE 382: note = THIOETH - position 382 is cysteine residue modified by thioether bond formation to maleimide-polyethylene glycol (PEG) linker
QVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRKELTKNQVSLTCLVKGFYPSDICVEWESNGQPENNYKTTPPVLKSDGSFFLYSKLTVDKSRWQQGNVFSCSVMHEALHNHYTQKSLSLSPG

SEQ ID NO: 36 - protein, 5 aa, synthetic construct
  REGION 1..5: note = Synthetic Construct
  SITE 1: note = FORMYLATION - Formylation of residue
  SITE 5: note = MISC_FEATURE - position 5 is lysine residue side chain modified through epsilon amide bond formation to a hydrolyzed maleimide-polyethylene glycol (PEG) linker
MIFLK

SEQ ID NO: 37-39 - skipped (3 entries)

SEQ ID NO: 40 - protein, 4 aa, synthetic construct
  REGION 1..4: note = Synthetic Sequence
  SITE 1: note = FORMYLATION - Formylation of residue
  SITE 4: note = MISC_FEATURE - position 4 is leucine residue C-terminally connected by amide bond formation to a polyethylene glycol (PEG) linker
MIFL

SEQ ID NO: 41 - protein, 4 aa, synthetic construct
  REGION 1..4: note = Synthetic Construct
  SITE 1: note = FORMYLATION - Formylation of residue
  SITE 4: note = MISC_FEATURE - position 4 is leucine residue modified by amide bond formation to a maleimide-polyethylene glycol (PEG) linker
MIFL

SEQ ID NO: 42 - skipped

SEQ ID NO: 43 - protein, 441 aa, synthetic construct
  REGION 1..441: note = Synthetic Construct
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 441 aa, synthetic construct
  REGION 1..441: note = Synthetic Construct
  SITE 155: note = THIOETH - position 155 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVCWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 441 aa, synthetic construct
  REGION 1..441: note = Synthetic Construct
  SITE 160: note = THIOETH - position 160 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGCLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 441 aa, synthetic construct
  REGION 1..441: note = Synthetic Construct
  SITE 257: note = THIOETH - position 257 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCCVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - protein, 441 aa, synthetic construct
  REGION 1..441: note = Synthetic Construct
  SITE 370: note = THIOETH - position 370 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPCDIAVEWESNGQPENNYKTTPPVLDSDGSFFLYSRLTVDKSRWQEGNVFSCSVMHEALHNHYTQKSLSLSLG

SEQ ID NO: 48 - protein, 441 aa, synthetic construct
  REGION 1..441: note = Synthetic Construct
  SITE 392: note = THIOETH - position 392 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPCLDSDGSFFLYSRLTVDKSRWQEGNVFSCSVMHEALHNHYTQKSLSLSLG

SEQ ID NO: 49 - protein, 441 aa, synthetic construct
  REGION 1..441: note = Synthetic Construct
  SITE 122: note = MISC_FEATURE - position 122 is cysteine residue modified by thioether bond formation to maleimide-polyethylene glycol (PEG) linker
  SITE 155: note = MISC_FEATURE - position 155 is cysteine residue modified by thioether bond formation to maleimide-polyethylene glycol (PEG) linker
  SITE 373: note = MISC_FEATURE - position 373 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPCVFPLAPCSRSTSESTAALGCLVKDYFPEPVTVCWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 50 - protein, 441 aa, synthetic construct
  REGION 1..441: note = Synthetic Construct
  SITE 122: note = THIOETH - position 122 is cysteine residue modified by thioether bond formation to maleimide-polyethylene glycol (PEG) linker
  SITE 160: note = THIOETH - position 160 is cysteine residue modified by thioether bond formation to maleimide-polyethylene glycol (PEG) linker
  SITE 373: note = THIOETH - position 373 is cysteine residue modified by thioether bond formation to maleimide-polyethylene glycol (PEG) linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPCVFPLAPCSRSTSESTAALGCLVKDYFPEPVTVSWNSGCLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 51 - protein, 449 aa, synthetic construct
  REGION 1..449: note = Synthetic Construct
  SITE 127: note = THIOETH - position 127 is cysteine residue modified by thioether bond formation to maleimide-polyethylene glycol (PEG) linker
  SITE 381: note = THIOETH - position 381 is cysteine residue modified by thioether bond formation to maleimide-polyethylene glycol (PEG) linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPEEKTISKQKGQPREPQVYTLPPSRDELTKNQVSLTCLVKGFYPSDICVEWESNGQPENNYKTTPPVLDSDGSFFLYSKLTVDKSRWQQGNVFSCSVMHEALHNHYTQKSLSLSPG

SEQ ID NO: 52 - protein, 329 aa, synthetic construct
  REGION 1..329: note = Synthetic Construct
  SITE 40: note = THIOETH - position 40 is cysteine residue modified by thioether bond formation to maleimide-polyethylene glycol (PEG) linker
ASTKGPSVFPLAPSSKSTSGGTAALGCLVKDYFPEPVTVCWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 329 aa, synthetic construct
  REGION 1..329: note = Synthetic Construct
  SITE 7: note = THIOETH - position 7 is cysteine residue modified by thioether bond formation to maleimide-polyethylene glycol (PEG) linker
  SITE 40: note = THIOETH - position 40 is cysteine residue modified by thioether bond formation to maleimide-polyethylene glycol (PEG) linker
ASTKGPCVFPLAPSSKSTSGGTAALGCLVKDYFPEPVTVCWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 326 aa, synthetic construct
  REGION 1..326: note = Synthetic Construct
  SITE 40: note = THIOETH - position 40 is cysteine residue modified by thioether bond formation to maleimide-polyethylene glycol (PEG) linker
ASTKGPSVFPLAPCSRSTSESTAALGCLVKDYFPEPVTVCWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 326 aa, synthetic construct
  REGION 1..326: note = Synthetic Construct
  SITE 45: note = THIOETH - position 45 is cysteine residue modified by thioether bond formation to maleimide-polyethylene glycol (PEG) linker
ASTKGPSVFPLAPCSRSTSESTAALGCLVKDYFPEPVTVSWNSGCLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 326 aa, synthetic construct
  REGION 1..326: note = Synthetic Construct
  SITE 7: note = THIOETH - position 7 is cysteine residue modified by thioether bond formation to maleimide-polyethylene glycol (PEG) linker
  SITE 40: note = THIOETH - position 40 is cysteine residue modified by thioether bond formation to maleimide-polyethylene glycol (PEG) linker
  SITE 258: note = THIOETH - position 258 is cysteine residue modified by thioether bond formation to maleimide-polyethylene glycol (PEG) linker
ASTKGPCVFPLAPCSRSTSESTAALGCLVKDYFPEPVTVCWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 57 - protein, 326 aa, synthetic construct
  REGION 1..326: note = Synthetic Construct
  SITE 7: note = THIOETH - position 7 is cysteine residue modified by thioether bond formation to maleimide-polyethylene glycol (PEG) linker
  SITE 45: note = THIOETH - position 45 is cysteine residue modified by thioether bond formation to maleimide-polyethylene glycol (PEG) linker
  SITE 258: note = THIOETH - position 258 is cysteine residue modified by thioether bond formation to maleimide-polyethylene glycol (PEG) linker
ASTKGPCVFPLAPCSRSTSESTAALGCLVKDYFPEPVTVSWNSGCLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 58 - protein, 212 aa, synthetic construct
  REGION 1..212: note = Synthetic Construct
RIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 59 - protein, 213 aa, synthetic construct
  REGION 1..213: note = Synthetic Construct
DIQMTQSPSSLSASVGDRVTITCSASSSVTYMYWYQRKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC